(19) United States
(12) Patent Application Publication
(10) Pub. No.: US 2018/0338971 A1
(43) Pub. Date: Nov. 29, 2018

(54) METHODS AND COMPOSITIONS FOR THE TREATMENT OF CUSHING'S SYNDROME USING 2S, 4R KETOCONAZOLE

(71) Applicant: STRONGBRIDGE IRELAND LIMITED, TREVOSE, PA (US)

(72) Inventor: Theodore Richard Koziol, Wynnewood, PA (US)

(21) Appl. No.: 16/007,298

(22) Filed: Jun. 13, 2018

Related U.S. Application Data
(63) Continuation of application No. 15/468,217, filed on Mar. 24, 2017, now abandoned.
Application No. 15/468,217 is a continuation of application No. PCT/US2015/051389, filed on Sep. 22, 2015.
(60) Provisional application No. 62/055,585, filed on Sep. 25, 2014.

(51) Int. Cl. A61K 31/496; G01N 33/74

(52) U.S. Cl. CPC A61K 31/496; G01N 2800/52; G01N 2800/04; G01N 33/743

(57) ABSTRACT

Methods for treating Cushing's Syndrome and other conditions associated with excessive cortisol production such as Cushing's Disease, ectopic adrenocorticotropic hormone secretion, ectopic corticotropin-releasing hormone secretion, adrenal-dependent Cushing's Syndrome, adrenal adenoma, adrenal autonomy, or any other condition associated with persistent or recurrent endogenous hypercortisolemia are provided. The methods include administration of a 2S,4R ketoconazole enantiomer substantially free of the 2R,4S enantiomer to patients according to a dosing titration schedule or at an effective amount. Treatment methods according to the invention may be safer and more effective than treatment using racemic ketoconazole.

### CROSS-REFERENCE TO RELATED APPLICATIONS

The present application is a Continuation of U.S. application Ser. No. 15/468,217 filed on Mar. 24, 2017 which is a Continuation of International Application No. PCT/US15/51389 filed on Sep. 22, 2015 which claims the benefit of U.S. Provisional Application No. 62/055,585 filed on Sep. 25, 2014, the contents of which are incorporated herein by reference.

### BACKGROUND

Endogenous Cushing's Syndrome (CS) is a rare but serious and potentially lethal endocrine disease caused by inappropriately excessive cortisol exposure to human organs. The incidence of CS has been estimated to be 2-3 cases per million population persons per year. The prevalence of the disease has been reported to be 30-60 cases per million. Patients with incompletely controlled disease are seriously ill and have at least a 5-fold increased rate of mortality, mainly due to metabolic and cardiovascular complications.

Treatment options for CS include surgery, radiation therapy and drug treatment. Medical treatment is used to suppress excessive cortisol production or activity and ameliorate its clinical manifestations prior to surgery or in patients awaiting the effects of radiation therapy as well as in patients where surgery is contra-indicated or a tumor cannot be found. As such, normalization of 24-hour urinary free cortisol (UFC) and normal serum or plasma cortisol levels from a low-dose overnight dexamethasone suppression test (DST) are considered adequate markers of disease remission. More recently, measurement of late night salivary cortisol levels is gaining clinical importance. Conversely, the persistence of high or only modestly reduced UFC levels argues for treatment failure.

Racemic ketoconazole (Nizoral®, the mixture of the two enantiomers 2S,4R and 2R,4S) is an approved antifungal agent that, at higher dosages, reduces adrenal steroid production via inhibition of multiple steroidogenic enzymes, e.g. 11β-hydroxylase, 17α-hydroxylase and aldosterone synthase. A direct effect on ectopic adrenocorticotropic hormone (ACTH) secretion by racemic ketoconazole has been seen. Because of these properties of ketoconazole, it is the most commonly used off-label drug for treatment of CS.

The single 2S,4R enantiomer of ketoconazole, is a drug for the treatment of cortisol hypersecretion in CS. The 2S,4R enantiomer of ketoconazole is isolated from racemic ketoconazole.

It is hypothesized that the 2S,4R enantiomer of ketoconazole may prove to be both safer and more efficacious than racemic ketoconazole. The 2S,4R enantiomer of ketoconazole has a significantly lower IC50 (50% inhibitory concentration) toward the key enzyme in cortisol synthesis (11-β-hydroxylase) and a significantly lower IC50 toward a key enzyme in cholesterol synthesis (14-α-demethylase) than does the other enantiomer, thus potentially allowing a lower dose of drug to achieve the same efficacy. (Rotstein, et al. “Stereoisomers of ketoconazole: preparation and biological activity.” J Med Chem 35(15): 2818-2, Welles, B., Glucocorticoids in type 2 Diabetes Mellitus and the Metabolic Syndrome. Drug Dev Res 67: 570-3 (2006), each of which is herein incorporated by reference). In rats, the 2S,4R ketoconazole enantiomer was more potent with respect to reducing corticosterone than the other enantiomers or racemic ketoconazole. It also appeared to reduce cholesterol more than the other enantiomer. (Schwartz, et al., Safety profile and metabolic effects of 14 days of treatment with DIO-902: results of a phase IIa multicenter, randomized, double-blind, placebo-controlled, parallel group trial in patients with type 2 diabetes mellitus. Clin Ther. 30(6): 1081-8 (2008)). In a clinical study, the 2S,4R ketoconazole enantiomer demonstrated a dose response reduction in C-reactive protein (CRP) levels compared to racemic ketoconazole, which showed no reduction in CRP levels. (Schwartz, et al. 2008).

### SUMMARY

Embodiments herein are directed to methods of administering 2S,4R ketoconazole according to a dosing titration schedule comprising one or more dosing levels as described in this document to a subject in need thereof. Some embodiments relate to methods of administering an effective amount of 2S,4R ketoconazole to a subject in need thereof. In some embodiments, the dosing titration schedule comprises administering a first dosing level of 2S,4R ketoconazole in an amount of about 150 to about 300 mg/day in 1 to 3 divided doses. In some embodiments, the first dosing level comprises administering a dose of 150 mg twice daily. In some embodiments, the first dosing level comprises administering a dose of 150 mg once daily. In some embodiments, the first dosing level comprises administering a dose of 100 mg three times daily. In some embodiments, the dosing titration schedule comprises administering a next dosing level when one or more of the following conditions are met: (1) mean 24-hour urinary free cortisol (UFC) levels are ≤ upper limit of the normal range (ULN) established for the assay being used at a central laboratory; (2) highest protocol-specified dose is reached; or (3) highest tolerated dose is reached. In some embodiments, the dosing levels are administered about 8 to about 22 days, preferably about 15 days. In some embodiments, the dosing titration schedule comprises administering a second dosing level of 2S,4R ketoconazole. In some embodiments, the second dosing level comprises administering 2S,4R ketoconazole in an amount of about 450 mg/day in 1 to 3 divided doses. In some embodiments, the second dosing level comprises administering 2S,4R ketoconazole in two divided doses of about 150 mg in a first dose and 300 mg in a second dose. In some embodiments, the dosing titration schedule comprises administering a third dosing level of 2S,4R ketoconazole. In some embodiments, the third dosing level comprises administering 2S,4R ketoconazole in an amount of about 600 mg/day in 1 to 3 divided doses. In some embodiments, the third dosing level comprises administering 2S,4R ketoconazole in two divided doses of about 300 mg in a first dose and 300 mg in a second dose. In some embodiments, the dosing titration schedule comprises administering a fourth dosing level of 2S,4R ketoconazole. In some embodiments, the fourth dosing level comprises administering 2S,4R ketoconazole in an amount of about 750 mg/day in 1 to 3 divided doses. In some embodiments, the fourth dosing level comprises administering 2S,4R ketoconazole in two divided doses of about 300 mg in a first dose and 450 mg in a second dose. In some embodiments, the dosing titration schedule comprises administering a fifth dosing level of 2S,4R ketoconazole. In some embodiments, the fifth dosing level comprises administering 2S,4R ketoconazole in an amount of about 900 mg/day in 1 to 3 divided doses. In some embodiments, the fifth dosing level comprises administering 2S,4R ketoconazole in two divided doses of about 450 mg in a first dose and 450 mg in a second dose. In some embodiments, the dosing titration schedule comprises administering a sixth dosing level of 2S,4R ketoconazole. In some embodiments, the sixth dosing level comprises administering 2S,4R ketoconazole in an amount of about 1050 mg/day in 1 to 3 divided doses. In some embodiments, the sixth dosing level comprises administering 2S,4R ketoconazole in two divided doses of about 450 mg in a first dose and 600 mg in a second dose. In some embodiments, the dosing titration schedule comprises administering a seventh dosing level of 2S,4R ketoconazole. In some embodiments, the seventh dosing level comprises administering 2S,4R ketoconazole in an amount of about 1200 mg/day in 1 to 3 divided doses. In some embodiments, the seventh dosing level comprises administering 2S,4R ketoconazole in two divided doses of about 600 mg in a first dose and 600 mg in a second dose.

Embodiments herein also relate to methods of treating Cushing's syndrome comprising administering to a subject in need thereof 2S,4R ketoconazole according to a dosing titration schedule comprising one or more dosing levels as described in this document. Some embodiments relate to methods of treating Cushing's Syndrome comprising administering to a subject in need thereof an effective amount of 2S,4R ketoconazole. Some embodiments relate to methods of treating a condition associated with the overproduction of cortisol comprising administering to a person in need thereof 2S,4R ketoconazole according to a dosing titration schedule described in this document. Some embodiments relate to methods of treating a condition associated with the overproduction of cortisol comprising administering to a subject in need thereof an effective amount of 2S,4R ketoconazole. In some embodiments, the condition associated with the overproduction of cortisol may be selected from Cushing's Disease, ectopic adrenocorticotropic hormone (ACTH) secretion, ectopic corticotropin-releasing hormone (CRH) secretion, adrenal-dependent Cushing's Syndrome, adrenal adenoma, adrenal autonomy, or any other condition associated with persistent or recurrent endogenous hypercortisolemia. In some embodiments, the effective amount of 2S,4R ketoconazole may be an amount per day of about 150 mg, 300 mg, 450 mg, 600 mg, 750 mg, 900 mg, 1050 mg, 1200 mg, or a range between any two of these values. In some embodiments, the dosing titration schedule comprises administering a first dosing level of 2S,4R ketoconazole in an amount of about 150 to about 300 mg/day in 1 to 3 divided doses. In some embodiments, the first dosing level comprises administering a dose of 150 mg twice daily. In some embodiments, the first dosing level comprises administering a dose of 150 mg once daily. In some embodiments, the first dosing level comprises administering a dose of 100 mg three times daily. In some embodiments, the dosing titration schedule comprises administering a next dosing level when one or more of the following conditions are met: (1) mean 24-hour urinary free cortisol (UFC) levels are ≤ upper limit of the normal range (ULN) established for the assay being used at a central laboratory; (2) highest protocol-specified dose is reached; or (3) highest tolerated dose is reached. In some embodiments, the dosing levels are administered about 8 to about 22 days, preferably about 15 days. In some embodiments, the dosing titration schedule comprises administering a second dosing level of 2S,4R ketoconazole. In some embodiments, the second dosing level comprises administering 2S,4R ketoconazole in an amount of about 450 mg/day in 1 to 3 divided doses. In some embodiments, the second dosing level comprises administering 2S,4R ketoconazole in two divided doses of about 150 mg in a first dose and 300 mg in a second dose. In some embodiments, the dosing titration schedule comprises administering a third dosing level of 2S,4R ketoconazole. In some embodiments, the third dosing level comprises administering 2S,4R ketoconazole in an amount of about 600 mg/day in 1 to 3 divided doses. In some embodiments, the third dosing level comprises administering 2S,4R ketoconazole in two divided doses of about 300 mg in a first dose and 300 mg in a second dose. In some embodiments, the dosing titration schedule comprises administering a fourth dosing level of 2S,4R ketoconazole. In some embodiments, the fourth dosing level comprises administering 2S,4R ketoconazole in an amount of about 750 mg/day in 1 to 3 divided doses. In some embodiments, the fourth dosing level comprises administering 2S,4R ketoconazole in two divided doses of about 300 mg in a first dose and 450 mg in a second dose. In some embodiments, the dosing titration schedule comprises administering a fifth dosing level of 2S,4R ketoconazole. In some embodiments, the fifth dosing level comprises administering 2S,4R ketoconazole in an amount of about 900 mg/day in 1 to 3 divided doses. In some embodiments, the fifth dosing level comprises administering 2S,4R ketoconazole in two divided doses of about 450 mg in a first dose and 450 mg in a second dose. In some embodiments, the dosing titration schedule comprises administering a sixth dosing level of 2S,4R ketoconazole. In some embodiments, the sixth dosing level comprises administering 2S,4R ketoconazole in an amount of about 1050 mg/day in 1 to 3 divided doses. In some embodiments, the sixth dosing level comprises administering 2S,4R ketoconazole in two divided doses of about 450 mg in a first dose and 600 mg in a second dose. In some embodiments, the dosing titration schedule comprises administering a seventh dosing level of 2S,4R ketoconazole. In some embodiments, the seventh dosing level comprises administering 2S,4R ketoconazole in an amount of about 1200 mg/day in 1 to 3 divided doses. In some embodiments, the seventh dosing level comprises administering 2S,4R ketoconazole in two divided doses of about 600 mg in a first dose and 600 mg in a second dose. In some embodiments, the effective amount of 2S,4R ketoconazole may be administered in one to three divided doses per day.

Embodiments herein are directed to methods of treating a condition associated with the overproduction of cortisol comprising administering 2S,4R ketoconazole according to a dosing titration schedule comprising one or more dosing levels as described in this document to a subject in need thereof. In some embodiments, the dosing titration schedule comprises administering a first dosing level of 2S,4R ketoconazole in an amount of about 150 to about 300 mg/day in 1 to 3 divided doses. In some embodiments, the first dosing level comprises administering a dose of 150 mg twice daily. In some embodiments, the first dosing level comprises administering a dose of 150 mg once daily. In some embodiments, the first dosing level comprises administering a dose of 100 mg three times daily. In some embodiments, the dosing titration schedule comprises administrating a next dosing level when one or more of the following conditions are met: (1) mean 24-hour urinary free cortisol (UFC) levels are ≤ upper limit of the normal range (ULN) established for the assay being used at a central laboratory; (2) highest protocol-specified dose is reached; or (3) highest tolerated dose is reached. In some embodiments, the dosing levels are administered about 8 to about 22 days, preferably about 15 days. In some embodiments, the dosing titration schedule comprises administering a second dosing level of 2S,4R ketoconazole. In some embodiments, the second dosing level comprises administering 2S,4R ketoconazole in an amount of about 450 mg/day in 1 to 3 divided doses. In some embodiments, the second dosing level comprises administering 2S,4R ketoconazole in two divided doses of about 150 mg in a first dose and 300 mg in a second dose. In some embodiments, the dosing titration schedule comprises administering a third dosing level of 2S,4R ketoconazole. In some embodiments, the third dosing level comprises administering 2S,4R ketoconazole in an amount of about 600 mg/day in 1 to 3 divided doses. In some embodiments, the third dosing level comprises administering 2S,4R ketoconazole in two divided doses of about 300 mg in a first dose and 300 mg in a second dose. In some embodiments, the dosing titration schedule comprises administering a fourth dosing level of 2S,4R ketoconazole. In some embodiments, the fourth dosing level comprises administering 2S,4R ketoconazole in an amount of about 750 mg/day in 1 to 3 divided doses. In some embodiments, the fourth dosing level comprises administering 2S,4R ketoconazole in two divided doses of about 300 mg in a first dose and 450 mg in a second dose. In some embodiments, the dosing titration schedule comprises administering a fifth dosing level of 2S,4R ketoconazole. In some embodiments, the fifth dosing level comprises administering 2S,4R ketoconazole in an amount of about 900 mg/day in 1 to 3 divided doses. In some embodiments, the fifth dosing level comprises administering 2S,4R ketoconazole in two divided doses of about 450 mg in a first dose and 450 mg in a second dose. In some embodiments, the dosing titration schedule comprises administering a sixth dosing level of 2S,4R ketoconazole. In some embodiments, the sixth dosing level comprises administering 2S,4R ketoconazole in an amount of about 1050 mg/day in 1 to 3 divided doses. In some embodiments, the sixth dosing level comprises administering 2S,4R ketoconazole in two divided doses of about 450 mg in a first dose and 600 mg in a second dose. In some embodiments, the dosing titration schedule comprises administering a seventh dosing level of 2S,4R ketoconazole. In some embodiments, the seventh dosing level comprises administering 2S,4R ketoconazole in an amount of about 1200 mg/day in 1 to 3 divided doses. In some embodiments, the seventh dosing level comprises administering 2S,4R ketoconazole in two divided doses of about 600 mg in a first dose and 600 mg in a second dose.

### BRIEF DESCRIPTION OF THE DRAWINGS

FIG. 1 is a timeline illustrating a single arm, open-label, dose titration study to assess efficacy, safety, tolerability, and PK of the 2S,4R ketoconazole enantiomer in subjects with CS.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “fibroblast” is a reference to one or more fibroblasts and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with elastin digest, can include, but is not limited to, providing an elastin digest into or onto the target tissue; providing an elastin digest systemically to a patient by, e.g., intravenous injection whereby the therapeutic reaches the target tissue; providing an elastin digest in the form of the encoding sequence thereof to the target tissue (e.g., by so-called gene-therapy techniques). “Administering” a composition may be accomplished by injection, topical administration, or by either method in combination with other known techniques. Such combination techniques include heating, radiation and ultrasound.

The term “animal” as used herein includes, but is not limited to, humans and non-human vertebrates such as wild, domestic and farm animals.

The term “Cushing's Syndrome” in this application refers to endogenous Cushing's syndrome which is caused by excessive endogenous cortisol production from various pathologies.

The term “Cushing's Disease” refers to endogenous Cushing's syndrome whereby the cause is a benign tumor in the pituitary gland causing excess ACTH production.

The term “improves” is used to convey that the present invention changes either the appearance, form, characteristics and/or the physical attributes of the tissue to which it is being provided, applied or administered. The change in form may be demonstrated by any of the following alone or in combination: enhanced appearance of the skin; increased softness of the skin; increased turgor of the skin; increased texture of the skin; increased elasticity of the skin; decreased wrinkle formation and increased endogenous elastin production in the skin, increased firmness and resiliency of the skin.

The term “inhibiting” includes the administration of a compound of the present invention to prevent the onset of the symptoms, alleviating the symptoms, reducing the symptoms, delaying or decreasing the progression of the disease and/or its symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. In part, embodiments of the present invention are directed to the treatment of cancer or the decrease in proliferation of cells.

A “therapeutically effective amount” or “effective amount” of a composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventive measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

In some embodiments, the compounds and methods disclosed herein can be utilized with or on a subject in need of such treatment, which can also be referred to as “in need thereof” As used herein, the phrase “in need thereof” means that the subject has been identified as having a need for the particular method or treatment and that the treatment has been given to the subject for that particular purpose.

### 1. Introduction

Endogenous Cushing's Syndrome (CS) is a rare but serious and potentially lethal endocrine disease caused by inappropriately excessive cortisol exposure to human organs. The incidence of CS has been estimated to be 2-3 cases per million population persons per year. The prevalence of the disease has been reported to be 30-60 cases per million. Patients with incompletely controlled disease are seriously ill and have at least a 5-fold increased rate of mortality, mainly due to metabolic and cardiovascular complications.

Treatment options for CS include surgery, radiation therapy and drug treatment. Medical treatment is used to suppress excessive cortisol production or activity and ameliorate its clinical manifestations prior to surgery or in patients awaiting the effects of radiation therapy as well as in patients where surgery is contra-indicated or a tumor cannot be found. As such, normalization of 24-hour urinary free cortisol (UFC) and normal serum or plasma cortisol levels from a low-dose overnight dexamethasone suppression test (DST) are considered adequate markers of disease remission. More recently, measurement of late night salivary cortisol levels is gaining clinical importance. Conversely, the persistence of high or only modestly reduced UFC levels argues for treatment failure.

Racemic ketoconazole (Nizoral®, the mixture of the two enantiomers 2S,4R and 2R,4S) is an approved antifungal agent that, at higher dosages, reduces adrenal steroid production via inhibition of multiple steroidogenic enzymes, e.g. 11β-hydroxylase, 17α-hydroxylase and aldosterone synthase. A direct effect on ectopic adrenocorticotropic hormone (ACTH) secretion by racemic ketoconazole has been seen. Because of these properties of ketoconazole, it is the most commonly used off-label drug for treatment of CS.

When patients are treated with ketoconazole, adrenal insufficiency is avoided by adjusting the dose to allow normal cortisol levels. Toxic side effects are rare with moderate doses but significant with the higher doses. The most frequent adverse effects of ketoconazole are nausea, vomiting, abdominal pain and pruritus. Abnormal liver function tests, primarily of the hepatocellular type, can occur. Rarely does severe hepatotoxicity occur. Early markers for these side effects are elevations in serum alkaline phosphatase, alanine aminotransferase (ALT), aspartate aminotransferase (AST), and bilirubin, which are monitored at regular intervals during treatment.

The single 2S,4R enantiomer of ketoconazole (sometimes referred to as COR-003), is a drug for the treatment of cortisol hypersecretion in CS. The 2S,4R ketoconazole enantiomer is isolated from racemic ketoconazole.

It is hypothesized that the 2S,4R ketoconazole enantiomer may prove to be both safer and more efficacious than racemic ketoconazole. The 2S,4R ketoconazole enantiomer has a significantly lower IC50 (50% inhibitory concentration) toward the key enzyme in cortisol synthesis (11β-β-hydroxylase) and a significantly lower IC50 toward a key enzyme in cholesterol synthesis (14-α-demethylase) than does the other enantiomer, thus potentially allowing a lower dose of drug to achieve the same efficacy. In rats, the 2S,4R ketoconazole enantiomer was more potent with respect to reducing corticosterone than the other enantiomers or racemic ketoconazole. It also appeared to reduce cholesterol more than the other enantiomer. In a clinical study, the 2S,4R ketoconazole enantiomer demonstrated a dose response reduction in C-reactive protein (CRP) levels compared to racemic ketoconazole, which showed no reduction in CRP levels.

Although the elevations in liver function tests (LFTs) associated with ketoconazole are generally modest in nature and are reversible following cessation of treatment, in rare cases (1 in 10,000 to 15,000 patients) severe hepatotoxicity may occur, and in extremely rare cases this adverse reaction may be irreversible and life-threatening. Preclinical data suggest that the 2S,4R ketoconazole enantiomer may have less risk on hepatic function than the racemic mixture. The 2S,4R ketoconazole enantiomer has a 12-fold higher IC50 toward CYP7A (IC50=2.4 μM) than does the other enantiomer, 2R,4S (IC50=0.195 μM). CYP7A is the first and rate-limiting enzyme in the liver resulting in 7α-hydroxylation of cholesterol and other oxysterols in the neutral pathway for production of bile acids. Inhibition of this enzyme can lead to functional cholestasis and consequent accumulation of potentially toxic metabolites such as bilirubin and xenobiotics such as ketoconazole itself.

A second property that suggests greater safety of the 2S,4R enantiomer compared to ketoconazole relates to the pharmacokinetics (PK) of the 2S,4R and 2R,4S enantiomers, which have been studied in humans after oral administration of ketoconazole or the 2S,4R ketoconazole enantiomer. The enantiomers in the racemic mixture are present in equal concentrations, but following administration of ketoconazole, blood concentrations of the 2S,4R enantiomer exceed those of the 2R,4S enantiomer by approximately 3-fold suggesting preferred extraction of the 2R,4S enantiomer by the liver. Reduced hepatic exposure to the 2S,4R enantiomer may be of value in reducing the liver function abnormalities observed following administration of racemic ketoconazole.

The 2S,4R ketoconazole enantiomer has been previously administered as a single chemical entity to healthy volunteers and human subjects with Type 2 Diabetes Mellitus (T2DM). Doses of the 2S,4R ketoconazole enantiomer over the range of 200 mg to 600 mg once daily (QD) for up to 14 days and 150 mg to 450 mg for up to 4 months were shown to be generally safe and well tolerated in all of these studies.

This study is the first clinical trial investigating the safety and efficacy of the 2S,4R ketoconazole enantiomer in subjects with endogenous CS. Where appropriate, the trial design has considered the reported experiences in dosing CS patients with racemic ketoconazole. Like ketoconazole, dosing of the 2S,4R ketoconazole enantiomer in CS patients needs to be highly individualized since initial clinical presentation and UFC levels are highly variable from patient to patient. Responsiveness to therapy is equally variable and results in a wide range of therapeutic doses which are tailored to the individual patient's medical situation. As such, the design of this study includes an initial Dose Titration Phase that will identify the optimal dose (i.e., therapeutic dose) for each patient, which will then be administered during the subsequent Maintenance Phase of the study. Improvement in clinical wellbeing and symptoms may be observed with UFC reductions, prior to full normalization of UFC. Clinical symptoms of intolerability are generally evident within 4 to 8 weeks of therapy and may begin during the titration phase of this study. This trial design will also allow for the identification of minimally active and maximally tolerated doses as well as the relationship between pharmacokinetic (PK) parameters of the 2S,4R ketoconazole enantiomer and its pharmacodynamic (PD) activity including safety.

### 1. Objectives

### 1.1 Primary Objective

To evaluate the efficacy of ascending doses of the 2S,4R ketoconazole enantiomer in subjects with elevated levels of cortisol due to endogenous CS by assessment of reduction in UFC levels (based on adequately collected 24-hour urine collections)
To identify the range of effective and safe doses of the 2S,4R ketoconazole enantiomer that reduce mean UFC levels to ≤ the upper limit of the normal range (ULN) of the assay at month 6 of the maintenance phase of dosing without a prior dose increase in that phase

### 1.2 Secondary Objectives

To identify the proportion of subjects with either or both of the following:
   mean UFC≤ULN or
   ≥50% reduction from baseline at month 6 of the maintenance phase
To characterize the changes in serum and late night salivary cortisol concentrations over 6 months of dosing in the maintenance phase
To assess the safety and tolerability of the 2S,4R ketoconazole enantiomer
To evaluate the resultant changes in protocol-defined metabolic endpoints associated with reductions in UFC levels
To assess the effects on clinical signs and symptoms of CS and the quality of life (QoL) measures obtained from the CushingQoL questionnaire
To assess changes in diabetic and antihypertensive therapies following treatment with the 2S,4R ketoconazole enantiomer
To evaluate the PK of the 2S,4R ketoconazole enantiomer in subjects with CS
To establish the dose (exposure) response relationship in reduction of UFC levels
To describe the dose (exposure) response relationship for safety of the 2S,4R ketoconazole enantiomer, as data permit

### 2. Endpoints

### 2.1 Primary Endpoint

Response to the 2S,4R ketoconazole enantiomer, defined as a reduction in mean 24-hour UFC levels to ≤ULN following 6 months of maintenance phase therapy without a prior dose increase during that phase

### 2.2 Secondary Endpoints

Response to the 2S,4R ketoconazole enantiomer, defined as mean UFC level ≤ULN at 1, 2, 3, 4, 5 and 6 months of a dosing in the maintenance phase and at 9 and 12 months in the extended evaluation phase
Response to the 2S,4R ketoconazole enantiomer, defined as UFC level ≤ULN or ≥50% reduction from baseline following 1, 2, 3, 4, 5 and 6 months of dosing in the maintenance phase and at 9 and 12 months in the extended evaluation phase
Mean UFC levels at 1, 2, 3, 4, 5 and 6 months of dosing in the maintenance phase and at 9 and 12 months in the extended evaluation phase, including percent change from baseline
Proportion of subjects with a reduction in UFC levels from baseline at 1, 2, 3, 4, 5 and 6 months of dosing in the maintenance phase and at 9 and 12 months in the extended evaluation phase
Shift in UFC normality at 1, 2, 3, 4, 5 and 6 months of dosing in the maintenance phase and at 9 and 12 months in the extended evaluation phase
Mean plasma and late night salivary cortisol levels at 1, 2, 3, 4, 5 and 6 months of dosing in the maintenance phase and at 9 and 12 months in the extended evaluation phase
Changes in clinical signs and symptoms (systolic and diastolic blood pressure, body weight/mass index, abdominal girth, body habitus and physical appearance, Cushing QoL questionnaire scores, etc.)
Changes in ambulatory blood pressure monitoring (ABPM) from baseline at each post-dose timepoint
Changes in biochemical markers from baseline at 3 and 6 months of dosing during the maintenance phase and at 9 and 12 months during the extended evaluation phase for the following: lipid profile (to include triglycerides, total HDL/LDL cholesterol, lipid ratios), fasting serum glucose levels, hemoglobin Alc (HbAlc), oral glucose tolerance test (pre-diabetics only), CRP, spot albumin/creatinine ratios (as a measure of microalbuminuria), ACTH, IGF-1 and testosterone concentrations (females only)
Changes in the doses of anti-diabetic and antihypertensive medications following 6 months of dosing with the 2S,4R ketoconazole enantiomer during the maintenance phase relative to baseline doses
PK parameters will be estimated (when possible) using population PK modeling and will include: clearance (CL/F), volume of distribution (V/F), absorption rate constant (Ka) with associated between subject variability where feasible, half-life (t1/2), area under the concentration time curve (AUC) and peak concentration (Cmax). Because the pharmacokinetics of the 2S,4R ketoconazole enantiomer are reported to change over time, time dependent changes in CL/F, AUC and t_{1/2} will be investigated, and if identified, will be incorporated into the model.
The PD model parameters will be estimated and will include: the 2S,4R ketoconazole enantiomer dose that produces maximal suppression of UFC (Imax), dose producing half maximal UFC suppression (IC50) and associated estimates of between subject variability. The range of UFC reduction by dose will be explored.
Safety evaluations will include clinical observations and adverse event (AE) reporting; physical examinations inclusive of vital signs (systolic and diastolic blood pressure, body weight, body mass index), hematology, chemistry panels (inclusive of expanded markers of liver function, serum cortisol and testosterone [males only] concentrations), dipstick urinalysis (microscopic evaluation, if dipstick positive) and IGF-1 concentrations.
Electrocardiograms (ECGs) will be evaluated. The relationship of QTc to dose (exposure) of the 2S,4R ketoconazole enantiomer will be evaluated, as data permit.

### 3. Study Design

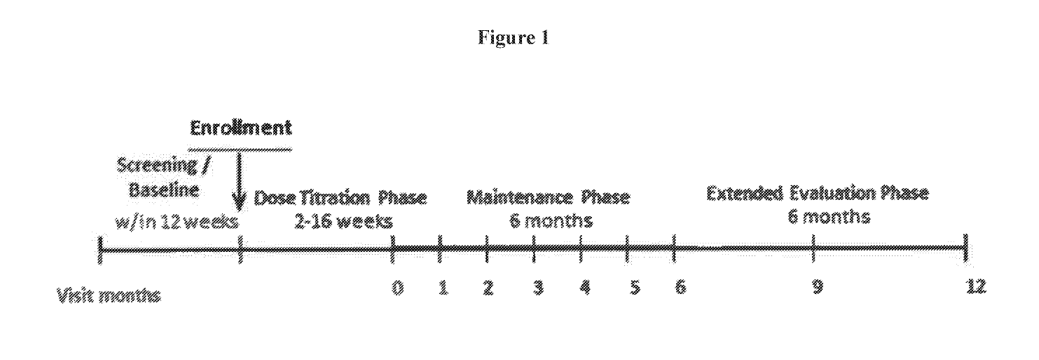

This will be a single arm, open-label, dose titration study to assess efficacy, safety, tolerability, and PK of the 2S,4R ketoconazole enantiomer in subjects with CS as illustrated in FIG. 1. The trial design will identify both the minimally effective and maximally tolerated doses of the 2S,4R ketoconazole enantiomer in this CS population. Following an initial screening period, this study will be conducted in 3 treatment phases as follows:
Dose titration phase: approximately 2 to 16 weeks to achieve an effective and tolerable maximum dose (the therapeutic dose)—see Section 3.1 for details;
Maintenance phase: 6 months of treatment at the therapeutic dose without a prior dose increase following the establishment of the appropriate dose identified in the titration phase;
Extended evaluation phase: 6 months of continued treatment after the maintenance phase; dose adjustments will be allowed as required for treatment

Efficacy will be assessed by measuring UFC levels at the times indicated in the Time and Events Table (Appendix A) and as described in Section 6.3.

Blood samples for the PK determination will be collected at the times indicated in the Time and Events Table (Appendix A) and as described in Section 6.4.

An independent Data Safety Monitoring Board (DSMB) will review the safety of the drug throughout the study. At a minimum, the DSMB membership will consist of a clinical pharmacologist with expertise in assessing safety issues in drug development, a liver specialist experienced in assessment of drug-induced liver injury (DILI), and a statistician. The full extent of responsibilities by the DSMB will be defined in their charter.

Subjects completing the 6-month maintenance phase of the study with the 2S,4R ketoconazole enantiomer will remain in the study for an additional 6 months for extended evaluations of the 2S,4R ketoconazole enantiomer treatment.

At the completion of the 6-month extended evaluation phase, subjects may return to their standard treatment and care, as applicable. 3.1 Dose Titration and Adjustment Criteria

The 2S,4R ketoconazole enantiomer will be administered twice daily (BID) according to the titration scheme in Table 1 until one of the following criteria has been met:
1. Mean 24-hour UFC levels are ≤ULN established for the assay being used at a central laboratory
2. Reached the highest protocol-specified dose
3. Reached the highest tolerated dose

TABLE 1
Dosing Titration Scheme

| Dose Level (DL) | Morning dosing | Evening Dosing |
|---|---|---|
| DL1 | 150 mg | 150 mg |
| DL2 | 150 mg | 300 mg |
| DL3 | 300 mg | 300 mg |
| DL4 | 300 mg | 450 mg |
| DL5 | 450 mg | 450 mg |
| DL6 | 450 mg | 600 mg |
| DL7 | 600 mg | 600 mg |

After establishing the baseline 24-hour UFC levels and confirmation of eligibility, each subject will begin dosing at dose level 1 (DL1) with an initial dose of 150 mg BID, taken once in the morning and once in the evening. Subsequent dose increases will be based on each subject's tolerability, assessment of UFC levels and safety data. The approximate interval between dose adjustments will be 15 (+7) days (including laboratory assay and reporting time).

At the discretion of the Investigator, subjects may be dosed on a TID schedule if the Investigator believes the subject will benefit from this dosing interval; however, the total daily dose at each dose level, as indicated in Table 1, should not be altered.

All subjects will be asked to collect 2 adequately collected 24-hour urine specimens starting at approximately Day 10 (+2 days) after the start of each dose level. They will bring their 2 urine collections to the clinic on approximately Day 12 (+2 days) for measurement of 24 hour UFC levels from each sample. Urine volume and creatinine will be measured as a marker of the adequacy of each collection (see Section 4.3.1). Subjects will continue on their current dose of the 2S,4R ketoconazole enantiomer until the UFC results have been obtained from the laboratory (within approximately 2-3 days). Based on their UFC results, subjects will be asked to return to the clinic to receive the first dose of drug for the next dose titration interval, as well as for scheduled assessments.

Dose titration and adjustment to achieve an effective dose of the 2S,4R ketoconazole enantiomer will be made as follows:
If UFC levels are >ULN of the assay for the mean of 2 adequate 24-hour urine collections, the dose should be increased to the next dose level, unless the highest dose has already been reached.
If the subject develops signs and/or symptoms of adrenal insufficiency (e.g., orthostatic hypotension, nausea, vomiting, abdominal pain) the Investigator may temporarily stop the medication to allow resolution of these symptoms and the dose will be reduced and restarted at a preceding dose level (see also Section 4.2.9.2). In the event that this observation is made at DL1, the subjects may receive a lower dose of 150 mg once daily (DL0) based on the medical discretion of the Investigator and agreement with the Sponsor. Subjects may resume the dose titration scheme after a dose reduction at the discretion of the Investigator and agreement with the Sponsor.
Subjects will have ECG evaluations at each dose level using the Spaulding Electrocardiograph device for collection of 5 minutes of continuous ECG. The dose of study medication will be reduced if the confirmed the 2S,4R ketoconazole enantiomer related QTc interval persistently increases to >500 msec or >60 msec from baseline. Please refer to Section 4.2.9.3 where guidance to the investigator on the assessment of the prolonged QTc interval is provided in detail. The Cortendo Medical Monitor should be contacted for guidance as necessary.

Adequate medical coverage should be provided at all times during the course of the study (including nights, weekends and holidays) to ensure that prompt safety decisions can be made and appropriate medical interventions provided. Subjects should be provided with instructions on how to access the medical staff regardless of day and time in order to obtain medical care.

Subjects will continue the process of dose titration until the therapeutic dose for the individual has been reached. When a subject achieves favorable UFC results (≤ULN for UFC) from two 24-hour urine samples, 2 additional 24-hour urine samples will need to be collected and returned to the site to confirm that the therapeutic dose has been reached and the subject can begin the Maintenance Phase of the study. Subjects should be contacted as soon as the results from the first two 24-hour urine samples are received to ask them to start the next two 24-hour urine collections.

The next visit will be considered Month 0 (i.e., Day 1 of Month 1) of the Maintenance Phase (see below for additional details) at which time, the subject will not be required to provide 24-hour urine samples.

A therapeutic dose for a subject will be considered established when mean UFC levels (determined from 4 complete 24-hour urine collections) are ≤ULN of the assay, or the maximum dose allowed has been reached. Four complete urine collections MUST be obtained at the maximum allowed dose or the therapeutic dose achieved for an individual subject.

Once the therapeutic dose has been reached and confirmed from a total of 4 complete 24-hour urine collections for UFC measurements, subjects will enter into the Maintenance Phase of the study. The very first visit during the Maintenance Phase will be called “Month 0” (MO) and will take place on the first day of Month 1 of the Maintenance Phase. After the MO visit, subjects will be asked to return to the clinic monthly for 6 months (i.e., end of Months 1, 2, 3, 4, 5, and 6) for efficacy (24 hour UFC measurements), clinical, safety, metabolic, Cushing QoL and PK assessments as outlined in the Time and Events Table (Appendix A). During the maintenance phase, doses may not be increased to maintain UFC levels at or below ULN of the assay, but doses can be reduced for safety reasons. At the discretion of the Investigator the subjects may resume dosing at the original maintenance phase dose. All dose adjustments must be documented. At the end of the 6-month maintenance period or at an early withdrawal visit, 4 complete 24-hour urine collections will again be obtained. In the continuing 6-month extended evaluation phase, subjects will return to the clinical site every 3 months for assessments, (i.e., during Months 9 and 12), and will have interim laboratory evaluations as indicated in the Time and Events Table (Appendix A).

Previously irradiated subjects must stop treatment with the 2S,4R ketoconazole enantiomer for ≥2 weeks after the end of the 6 month maintenance phase and provide 4 complete 24-hour urine collections for UFC measurements. They may subsequently restart therapy and continue into the 6-month extended evaluation phase at the discretion of the Investigator.

### 3.1.1 Dose Escalation Above 600 mg Total Daily Dose

The 2S,4R ketoconazole enantiomer has only been dosed up to 600 mg/day in diabetic subjects. The safety in subjects at doses beyond 600 mg/day is unknown. For this reason, subjects that reach total daily doses of >600 mg/day (i.e., doses of 750, 900, 1050, and 1200 mg/day) must be monitored more closely in order to ensure proper evaluation of the safety of doses at these levels. Subjects will be advised to contact the Investigator immediately in the event of developing symptoms in view of the dose escalation.

In addition to the assessments that will be carried out during dose escalation for doses ≤600 mg/day, for each dose escalation beyond 600 mg/day, subjects will be asked to return after 4-7 days and again at 14-16 days after dose escalation for evaluation of symptom-specific AEs (see Section 11.2.2), vital signs, routine safety laboratory assessments (including LFTs), and serum cortisol levels as outlined in Time and Events table (Appendix A). The results of the assessments at 14-16 days may serve as baseline values for the next dose level.

The results of relevant assessments should be available promptly in order for the Investigator to make decisions for the safety of the subject.

Reports of Serious Adverse Events (SAEs) experienced at these higher doses will be shared with all investigators regularly during the study.

### 3.2 Stopping Criteria

Dosing with the study medication will cease due to any of the following observations:
Intolerability to the study medication based on the subjects' symptoms in accordance with the Investigator's medical judgment
Lack of any measurable response at the maximally tolerated dose
QTc prolongation as specified in Section C at 150 mg/day
Liver function test abnormalities as specified in D
Adrenal insufficiency at the lowest dose of the 2S,4R ketoconazole enantiomer (150 mg daily)—See also Section D for more details.
Any withdrawal criteria specified in Section B

The cause for withdrawal will be documented and appropriately captured in the database. Withdrawn subjects, due to any of the above criteria, must receive appropriate follow up medical care. The Investigator will explain all other treatment options available in that country to them, and they will be promptly referred back to their endocrinologist (if not the Investigator) for further management according to the local standard of care, and based on their preceding medical history.

### Subject Selection and Withdrawal Criteria

Cortendo will review each subject's enrollment criteria to ensure that subjects meet the eligibility criteria.

A. Inclusion Criteria

Subjects eligible for enrollment in the study must meet all the following criteria:
1. Male or female ≥18 years of age
2. Confirmed diagnosis of persistent or recurrent CS (with or without therapy) or newly diagnosed disease, if they are not candidates for surgery. CS will be defined according to the criteria in the guidelines for diagnosis of CS (2008). Previous medical records will be collected and used to support the diagnosis. The diagnostic criteria for appropriateness of inclusion of each subject into the study will be reviewed by the Medical Monitor.
   Subjects MUST have elevated 24-hour UFC levels ≥1.5×ULN of assay based on a minimum of 4 measurements from adequately collected urine. Urine may be collected on sequential days.
   Diagnosis of the disease will also need to be based on the association of clinical features of endogenous CS (see Appendix), review of past medication history, excluding exogenous sources of glucocorticoids, and abnormal values from one of the following tests:
      i. Abnormal DST: Elevated 8 AM serum cortisol ≥1.8 μg/dL (50 nmol/L) after 1 mg dexamethasone orally at 11 PM the evening prior (if not conducted already in the diagnostic workup of the subject within the previous 2 months; previous test results and details of conduct will need to be available)
      ii. Elevated late night salivary cortisol concentrations (at least 2 measurements)>ULN at screening NOTE: For subjects with estimated glomerular filtration rate (eGFR as determined by Modified Diet in Renal Disease MDRD equation)>40 and <60 mL/min/1.73 m² in addition to meeting the UFC criteria, late night salivary cortisol test results (≥2 measurements) MUST also demonstrate evidence of CS.
   For subjects with Cushing's disease who have already undergone surgery, a minimum of 3 months should have elapsed before the subject can be deemed a surgical failure.
3. Previously irradiated subjects will be allowed as long as the radiation treatment occurred >4 years ago and they have not exhibited evidence for improvement in their underlying Cushing's disease for 6 months. The total number of previously irradiated subjects enrolled in this study will not exceed 10.
   In the vast majority of subjects treated with radiation, efficacy is observed in <4 years.
4. Subjects who refuse surgery or in whom surgery will be delayed beyond the projected duration of this study will be permitted to participate.
5. Confirmed diagnosis of persistent or recurrent endogenous hypercortisolemia as defined by elevated 24-hour UFC levels on repeated determinations (described in Inclusion #2) caused by either ACTH-dependent or ACTH-independent etiologies.
   Cushing's Disease (CD) which is specifically defined as mean 24-hour UFC level of ≥1.5×ULN on repeated determination, morning plasma corticotropin (ACTH) level of 5 ng/L (1.1 nmol/L) or more, a confirmed pituitary source of CD (documentation of ACTH immunoreactivity on pathological evaluation), and as determined by medical records (including surgical reports and pituitary imaging scans), or bilateral inferior petrosal sinus sampling (BIPSS) with a central to peripheral ratio of >2 before or >3 after corticotropin-releasing hormone (CRH) administration)
   Ectopic ACTH secretion, not of pituitary origin
   Ectopic CRH secretion
   Adrenal-dependent CS (adrenal adenoma, adrenal autonomy)
   Etiology unknown
6. Subjects whose CS is due to Cushing's disease with or without radiographically visible adenomas may be enrolled based on a pituitary magnetic resonance imaging (MRI) scan may be enrolled as long as biochemical criteria in Inclusion Criteria #5 are met.
7. Subjects on treatment for CS for whom treatment has been inadequate or not well tolerated must agree to the following minimum washout periods as determined by the nature of their treatment before baseline assessments are performed for participation in this study:
   Inhibitors of steroidogenesis (including ketoconazole): 2 weeks
   Dopamine agonists: bromocriptine (2 weeks), cabergoline (8 weeks)
   Octreotide acetate LAR and lanreotide Autogel®: 12 weeks
   Lanreotide SR/long-acting pasireotide: 8 weeks
   Octreotide acetate (immediate release formulation) or short-acting pasireotide: 1 week
   Mifepristone (RU 486): 4 weeks
8. Subjects on megesterol acetate (medroxyprogesterone acetate) must agree to a wash out of ≥6 weeks prior to receiving the first dose of the study medication
9. Subjects with impaired fasting glucose, diabetes and/or hypertension may be enrolled
10. A female is eligible to enter and participate in the study if she is of:
   Non-child bearing potential (i.e. physiologically incapable of becoming pregnant, including any female who is post-menopausal or surgically sterile). Surgically sterile females are defined as those with a documented hysterectomy and/or bilateral oophorectomy or tubal ligation. Post-menopausal females are defined as being amenorrhoeic for greater than 1 year with an appropriate clinical profile, e.g. age appropriate, >45 years, in the absence of hormone replacement therapy. However in questionable cases, a blood sample with FSH>40 MIU/ml and estradiol <40 pg/ml (<140 pmol/L) is confirmatory.
   OR
   Child bearing potential, has a negative pregnancy test at screening, and agrees to one of the following acceptable contraceptive methods used consistently and correctly (i.e. in accordance with the approved product label and the instructions of the physician for the duration of the study—screening to follow-up contact):
      Complete abstinence from intercourse; or
      Male partner is sterile prior to female subject entry into the study, and this male partner is the sole partner for that subject; or
      Implants of levonorgestral inserted for at least 1 month prior to the study medication administration but not beyond the third successive year following insertion; or
      Oral contraceptive (combined or progestogen only) administered for at least one monthly cycle prior to study medication administration; or
      Double barrier method: condom or occlusive cap (diaphragm or
      cervical/vault caps) plus spermicidal agent (foam/gel/film/cream/suppository); or
      An intrauterine device (IUD); or
      Estrogenic vaginal ring; or
      Percutaneous contraceptive patches
11. 12-lead ECGs show no acute ischemia or clinically significant abnormality needing medical intervention.
12. Ability to comprehend and comply with procedures.
13. Agree to commit to participate in the current protocol.
14. Subjects provide written informed consent prior to any study procedures being performed (all subjects should be able to understand the informed consent form and any other documents that subjects are required to read).

### 3.2 Exclusion Criteria

Subjects will be excluded from the study if any of the following criteria are met:
1. De novo Cushing's disease AND a candidate for pituitary surgery
   If surgery is to be delayed for >6 months, subjects may be allowed to participate in the trial while awaiting surgery, but must agree to complete this study prior to surgery.
2. Subjects treated with radiation within the previous 4 years or >4 years and with evidence of improvement in their disease within 6 months prior to Screening.
   In the majority of subjects treated with radiation, efficacy is observed in <4 years.
3. Subjects who have been treated with mitotane within 6 months of screening or have levels exceeding 5 μg/mL.
4. Pseudo-Cushing's syndrome based on clinical assessment of the investigator. Appendix D describes a number of conditions in which elevated cortisol levels may be observed in the absence of Cushing's syndrome, sometimes referred to as Pseudo-Cushing's syndrome.
5. Subjects with adrenal carcinoma.
6. Body habitus preventing repeated venipuncture as required by protocol.
7. Subject is currently in another study or has received any investigational treatment (drug, biological agent or device) within 30 days or 5 half-lives of screening, whichever is longer.
8. Male and female subjects with QTc interval of >470 msec.
9. History of Torsades des Pointes or ventricular tachycardia or ventricular fibrillation or history of prolonged QT syndrome (including family history) or use of medications resulting in QT/QTc prolongation or hypokalemia <3.0 meq/L.
10. Subjects with a non-endogenous source of hypercortisolemia such as exogenous source of glucocorticoids or therapeutic use of ACTH.
11. History of malignancy, other than thyroid, early stage prostate, squamous cell and basal cell carcinoma, within 3 years prior to Screening. Subjects with history of carcinoma must have a life expectancy of >1 year and must be on stable doses of their specific therapies. Subjects with early stage prostate cancer undergoing no treatment due to low grade potential may be enrolled.
12. Diagnosis of HIV.
13. History of persistent uncontrolled hypertension (>180/120 mmHg) despite medical intervention.
14. Subjects with hypercholesterolemia who are on current atorvastatin or simvistatin and not willing or unable to change to alternative therapies as noted (pravastatin, fluvastatin, and rosuvastatin) within 2 weeks of study screening. Subjects may sign informed consent and then immediately begin screening assessments once they have discontinued the prohibited statin.
15. Subjects requiring repeated frequent hospitalizations for management of hyperglycemic episodes.
16. Subjects with decreased renal function as defined by eGFR<40 mL/min/1.73 m², using MDRD equation for estimating renal function (eGFR).
17. Any other clinically significant medical condition, as determined by the Investigator that precludes enrollment and participation in the study through completion (for example, New York Heart Association (NYHA) class III or IV congestive heart failure).
18. Known hepatic disease, other than mild to moderate hepatic steatosis consistent with fatty infiltration (non-alcoholic steatohepatitis [NASH]).
19. History of recurrent gall stone attacks or pancreatitis.
20. Positive for hepatitis B surface antigen (HbsAg) or positive hepatitis C test.
21. Liver function tests (LFT) must not be above the following cut-offs at screening: ALT and/or AST>3.0×ULN, GGT>2×ULN, and total bilirubin >2×ULN. If all LFTs are within normal limits (WNL) and total bilirubin is elevated, examination of direct and indirect bilirubin may be conducted. Subjects with indirect total bilirubin up to 3×ULN are presumed to have Gilbert's syndrome and may be enrolled if all other LFTs are WNL.
22. History of documented clinically significant liver function abnormalities requiring drug discontinuation on ketoconazole or closely related chemical analogues (for example, itraconazole).
23. Presence of any other clinically significant medical condition, as determined by the Investigator that would preclude the subject from being able to follow instructions or to perform the necessary procedures (for example, psychiatric instability or severe disability).
24. Compression of the optic chiasm.
25. Abnormal free T4. Subjects with TSH<LLN and normal free T4 are permitted to participate in the study.
26. Subjects who have a history of alcohol or drug abuse in the 6 month period prior to enrollment.
27. The subject is currently taking any H2 receptor antagonists, proton-pump inhibitors, or sucralfate (which inhibit absorption of the 2S,4R ketoconazole enantiomer). A list of orally acceptable antacids (for example, Mylanta and Maalox) will be provided, and can only be taken a minimum of 2 hours after dosing of the 2S,4R ketoconazole enantiomer.
28. The subject is receiving the following concomitant therapies:
   Weight loss medications (prescription or over the counter)
   Acetaminophen >3 g total daily dose (due to increased hepatotoxicity)
   Co-administration of strong inducers or inhibitors of CYP3A4 enzyme system that may interfere with the 2S,4R ketoconazole enantiomer and cannot be discontinued prior to dosing (see Section 10.2 for further details)
   Statins other than pravastatin, fluvastatin and rosuvastatin
   The following herbal medicines are prohibited: St John's Wort, echinacea, gingko, goldenseal, yohimbe, red rice yeast, danshen, silybum marianum, Asian ginseng, schissandra sphenanther, shankhapushi, and Asian herb mixture (Xiao chai hu tang and Salboku-to).
   Topical or inhaled steroids.
   Carbamazipine, fenofibrate, carbenoxolone.
   Excessive ingestion of genuine licorice.
29. Pregnant women or lactating women, or women of child bearing potential not practicing a medically acceptable method for birth control or sexually active men not using contraception. Women must agree to continue to use an acceptable method of contraception and men must use condoms, if sexually active. All forms of contraception, used by male and female subjects, must be used during the course of the study and for 2 weeks after the study is completed.
30. Any other condition which would jeopardize the risk of subject participation in the trial in the opinion of the Investigator

B. Withdrawal Criteria

Reasons for withdrawal (subject refuses to return for any remaining study visits) or discontinuation (subject who prematurely stops the treatment) at any time during the study may include, but are not limited, to the following:
For safety reasons, either at the discretion of the Investigator or at the subject's request (see also Section 3.2, Stopping Criteria).
For protocol violations at the discretion of the Sponsor.
Due to concomitant therapy that could interfere with the results of the study (the Investigator will report all such information on the CRFs and decide, in accordance with the Sponsor, whether the subject is to be withdrawn).

All premature discontinuations and their causes must be carefully documented by the Investigator on the CRF, and, if need be, on the AE page.

If, for any reason, a subject is withdrawn before completing the study, the reason for termination will be entered in the CRF. All data gathered on the subject prior to termination will be made available to the Sponsor. Subjects not completing the entire study should be fully evaluated when possible, including completing the follow-up visit.

Additional subjects may be enrolled to account for withdrawn subjects prior to completion of the maintenance phase of the study to maintain the sample size for analysis. Withdrawn subjects and subjects who reach the highest tolerated dose without a response will be considered non-responders.

All withdrawn subjects will be asked to report to the clinic for a follow-up visit at approximately 2 weeks after receiving their last dose of the study medication. All AEs will be followed until resolution or at a minimum of 30 days after the last dose of the study medication. All assessments at the follow-up visit are listed in the Time and Events Table (Appendix A).

C. Withdrawal Due to QTc Interval Prolongation

Administration of the study drug will cease and the subject will be withdrawn upon persistent and confirmed the 2S,4R ketoconazole enantiomer-related QTc interval prolongation >500 msec observed at the lowest 2S,4R ketoconazole enantiomer dose (150 mg/day) ONLY. At other doses, persistent QTc prolongations due to the 2S,4R ketoconazole enantiomer can be managed by dose reduction. Elevation of QTc>500 msec will be evaluated using the 5-minute Spaulding Electrocardiograph device. The Investigator should consider the possibility of concomitant medications being used that may result in prolongation of the QTc interval either directly or through interference with the metabolism of the 2S,4R ketoconazole enantiomer, recent food ingestion (temporary QTc elevation), and electrolyte abnormalities (particularly serum calcium, magnesium and potassium) as causative or contributory to the QTc prolongation and should be assessed prior to discontinuation of the 2S,4R ketoconazole enantiomer. Interfering concomitant medications with this potential are contraindicated in co-administration with the 2S,4R ketoconazole enantiomer and must be stopped. Please see Section 4.2.9.3 for guidance on the assessment of QTc interval prolongation. Regardless of seriousness or causality, instances of persistent QTc prolongation should be reported to the Sponsor's designate safety group within 24 hours, in the same manner as SAEs. The Investigator should contact the Cortendo Medical Monitor for guidance as needed as well.

If QTc prolongation is observed, an additional PK sample should be collected as close to the time of the event as possible.

D. Withdrawal Due to Liver Function Tests (LFTs)

LFTs will be measured at every study visit. The recommendations within the FDA Guidance on Drug-Induced Liver Injury: Premarketing Clinical Evaluation (May 2009) have been adopted for use in this protocol to address abnormal values. LFTs will be measured immediately if subjects develop signs and symptoms suggestive of hepatic dysfunction (any of the following: nausea, anorexia, fever, fatigue, right upper quadrant discomfort, pruritus, dark urine or acholic stool). Nausea, anorexia, and fatigue are nonspecific symptoms and may be caused by hypocortisolemia; however, appropriate medical evaluation of such symptoms must include assessment of LFTs. Other labs to assess causality (hepatitis B, C, etc.) and consultation with a liver specialist must be obtained upon confirmation of the LFT elevation (which was a cause for concern prior to a planned withdrawal of the subject). Appropriate medical evaluations and interventions should be implemented based on the clinical presentation of the subject, including appropriate diagnostic imaging procedures. All such medical interventions will be recorded in the CRF. While LFTs may be assayed at a local lab for immediate medical intervention, simultaneous samples must be sent to the central laboratory for DSMB assessment to ensure consistency of assay and interpretation.

Subjects withdrawn due to LFT abnormalities must be followed until normalization. Subjects may be rechallenged after resolution of LFT abnormalities, at the discretion of the Investigator AND with approval by the Cortendo Medical Monitor.

The following levels obtained at any time during the study will guide the investigators in study drug cessation/early termination:
ALT or AST>8×ULN
ALT or AST rises to >5×ULN in <4 weeks or persists for >2 weeks
ALT or AST>3×ULN and total bilirubin >2×ULN or INR>1.5
ALT or AST>3×ULN with new onset of or worsening of fatigue, nausea, vomiting, fever, rash or eosinophilia, in the absence of other evident cause
AP>2.0×ULN (confirmed as liver in origin by elevated gamma-glutamyl transpeptidase [GGT])
Total bilirubin >2×ULN (except for subjects enrolled with presumed Gilbert's syndrome)
Signs and/or symptoms suggestive of hepatic dysfunction (any of the following: nausea, anorexia, fever, fatigue, right upper quadrant discomfort, pruritus, dark urine or acholic stool) coupled with ALT and/or AST>3×ULN and/or AP>2×ULN, and/or total bilirubin >2×ULN (unless Gilbert's syndrome has been noted)

Subjects with ALT and/or AST>3×ULN or AP>2×ULN (of liver origin) or total bilirubin >2× (except for subjects enrolled with presumed Gilbert's syndrome) at any visit will undergo serial repeat LFT and International Normalized Ratio (INR) evaluations. The first repeat LFT should occur as soon as possible after initial determination to confirm the observation and thereafter at 3 to 4 day intervals or as the clinical situation dictates. Appropriate medical evaluations and interventions should be implemented based on the clinical presentation of the subject. Once results of these tests are available, the Investigator will consult with the Sponsor Medical Monitor. If the LFTs return towards baseline, in the opinion of the Investigator and Medical Monitor, serial measurements will be discontinued and the subject will continue for the remainder of the trial.

If on serial measurements and in the absence of clinical signs or symptoms, the ALT and/or AST continue to rise (but AP and total bilirubin do not exceed the cut-offs for study drug cessation/early termination), subjects will continue on study drug and protocol unless the ALT and/or AST levels are >8×ULN. If the ALT and/or AST levels exceed these levels, the subject should stop taking the study medication immediately.

Medical evaluation should be undertaken as appropriate to the signs and symptoms manifested by the subject and recorded in the CRF.

### 4. Study Assessments and Procedures

The parameters for each assessment are listed and described in this section. The exact timings of each assessment are provided in the Time and Events Table (Appendix A). Detailed procedures for obtaining each assessment are provided in the Study Procedures Manual (SPM).

The timing and number of planned study assessments, including safety, PK, and efficacy assessments may be altered during the course of the study based on newly available data to ensure appropriate monitoring. The change in timing for any planned study assessments must be approved and documented by Cortendo, but this will not constitute a protocol amendment. The addition of any new study assessments to the protocol would constitute an amendment.

In addition to the protocol-specified procedures, at any time during the study appropriate medical evaluations and interventions should be implemented, as necessary, based on the clinical presentation of the subject according to standard of care. Changes to the medical conditions, history and medications and all other medical interventions will be documented in the CRFs.

The study will consist of the following phases with approximate timings:
Screening phase (within approximately 12 weeks of first dose)
Dose titration phase (approximately 2-16 weeks)
Maintenance phase (6 months)
Extended evaluation (6 months)

Baseline evaluations should be conducted as close as possible to the initiation of dosing in the Dose Titration Phase, but only after subjects have undergone a sufficient washout period from previous CS therapies (see Section 8.2), if applicable. It is recommended that the 4 required 24-hour urine samples be collected for baseline UFC determinations prior to performing the remaining baseline assessments. In addition, it is recommended that the 4 urine samples be collected on sequential days and brought to the clinic within 1-2 days of the last collection since UFC is only stable for 7 days under refrigerated storage conditions. Results from the baseline assessments are necessary in order to determine if the subject remains eligible for participation in the study. Please see the SPM for details on urine collections.

### 4.1 Demographic/Medical History Assessments

The following demographic parameters will be captured at the Screening visit:
Date of birth
Gender
Race and ethnicity

Medical and medication history will be assessed and collected as related to the Eligibility Criteria in Section A and will include the following, as available:
An effort will be made to collect the following information for documentation of medical history as part of this study:
   Documentation of diagnosis of CS, including evaluations for CS for the previous 6 months, e.g., levels of UFC, late night salivary and/or serum cortisol, DST results, BIPSS results and diagnostic imaging results, etc.
   Documentation of associated metabolic complications (e.g., diabetes, hypertension, osteoporosis, and reproductive dysfunction)
   Documentation on prior management of CS for the previous 3 months, including medications (capturing doses, duration of treatment, time of discontinuation and reason for discontinuation of medication) and any surgical or radiation therapies
Available clinical laboratory data for the previous 3 months; specifically, data related to the following parameters: LFTs (ALT, AST, AP, direct and total bilirubin), HbAlc, fasting serum glucose, cholesterol/triglyceride panel, CRP, urinary albumin/creatinine ratio, testosterone and IGF-1 concentrations
Available blood pressure data for the previous 3 months
Medication history (all medications, including any non-prescription medications, other than those to treat CS specifically) taken within 3 months prior to Screening with particular attention to blood pressure, anti-diabetic and cholesterol lowering drugs
History of drug and alcohol use

### 4.2 Safety Assessments

Subject eligibility must be evaluated at screening, baseline and on an ongoing basis throughout the study as listed in the Time and Events Table (Appendix A). Subjects must be medically managed for all signs and symptoms of CS throughout the study at unscheduled visits as deemed appropriate by the Investigator according to the standard of care for treatment of CS and its attendant conditions. All such assessments will be recorded in the CRF.

### 4.2.1 Physical Exam and Assessment of Clinical Signs and Symptoms of CS

Full physical exams will be performed by a physician at the times indicated in the Time and Events Table (Appendix A). The physical examinations will be inclusive of all body systems, and should include height (cm) and weight (kg) and signs and symptoms of CS at all scheduled study visits. Abdominal girth will be measured in triplicate at the times indicated in the Time and Events table and as described in the SPM. Body mass index (BMI) will be calculated based on data entered into the CRF during that visit.

As part of the physical exam visits, both the subjects and the examining physician will be asked to complete questionnaires related to the assessment of physical signs of CS using a visual analog scale (VAS) or other grading instruments (Appendix C). The results from these questionnaires will be used to quantify changes in signs of CS. Details regarding these assessments will be provided in the SPM.

Standard photographs to document signs of CS will be taken for consenting subjects at the times indicated in the Time and Events Table (Appendix A). Photographs will only be taken for subjects who consent to photography. At a minimum, the following signs will be evaluated using photographs: facial rubor, hirsutism, striae, bruising, supraclavicular and dorsal fat pad, and general body habitus. Formal assessment tools will be provided by which each subject will be evaluated in a standardized fashion.

The identity of the subjects will be appropriately masked on the images at the clinical site. Pictures will include close-up of the face as well as frontal and sagittal views of the neck, trunk, arms and legs, as detailed in the SPM. The eyes and any specific identifying characteristics of the subject (tattoos) can be masked as long as the masking does not interfere with the evaluation of the body habitus. Subjects may wear underwear for the photographs. Examples of images will be provided to ensure consistency of the photographic images. Details will be provided in the SPM. A sufficient number of photographs, frontal and lateral views, from the shoulders upwards will be taken to assess facial plethora, supraclavicular and dorsal fat pads. A sufficient number of photographs of frontal and dorsal views of the trunk with subjects in standing position will be taken to assess striae, bruising, hirsutism and general body habitus. In total, approximately 7-10 photographs will be taken. Female subjects will be asked not to shave during 3 weeks prior to baseline and the visits at which photographs will be taken as indicated in the Time and Events Table (Appendix A).

Physical exams will be performed as indicated at every study visit and recorded. Symptom-driven physical exams will be performed by the Investigator as clinically required and recorded as part of the evaluation of an AE.

All subjects, regardless of dose, will be evaluated for AEs at predose (See Section 11.2.1 for disease-related AEs and Section 11.2.2 for symptom-specific AEs). Specific targeted symptoms and signs will be evaluated throughout the study for all subjects. At doses exceeding 600 mg/day, subjects will be assessed for specific symptoms at 4-7 days and 14-16 days after each dose escalation in addition to pre-dose.

### 4.2.2 Vital Signs

Vital sign measurements will include temperature, systolic and diastolic blood pressure (SBP, DBP) and heart rate (HR) at baseline. Blood pressure measurements and resting HR will also be measured at each visit throughout the study (see Time and Events Table in Appendix A).

For proper measurement of blood pressure, the following procedures should be followed:
Use of an appropriately sized cuff for the size of the subject's arm circumference should be utilized to minimize inaccurate readings.
No smoking or exercise for at least 30 minutes before a blood pressure measurement
Subjects should sit in a chair with a back support and the arm supported at heart level with feet flat on the floor. The subject should void prior to the measurement.
Blood pressure measurements will be measured in triplicate over a minimum of approximately 10 minutes after the subject has rested in a sitting position for at least 10 minutes. The 3 measurements will each be recorded in the CRF and a mean value for that visit will be calculated for reporting.

Vital sign measurements must be repeated if clinically significant or machine/equipment errors occur. Out of range blood pressure or HR measurements will be repeated at the Investigator's discretion. Any confirmed, clinically significant vital sign measurements must be recorded as AEs.

Ambulatory blood pressure monitoring for 24 hours will be evaluated at baseline and during the Maintenance phase of the study (0, 3 and 6 months). Details for the assessments will be provided in the SPM.

Guidelines for classification for blood pressure levels and recommendations for follow-up are provided in Appendix C features of Cushing's syndrome. Signs and symptoms of CS include the features listed in the table below; however, they should not be limited to the list. Additional signs and symptoms may be determined by the investigator, as appropriate.

Clinical Features of Cushing's Syndrome^{a}

| | | |
|---|---|---|
| Symptoms | Signs | Overlapping Conditions |

Features that best discriminate CS; most do not have a high sensitivity

Easy bruising
Facial plethora
Proximal myopathy (or proximal muscle
weakness)
Striae (especially if reddish purple and
>1 cm wide)

CS features in the general population that are common and/or less discriminatory

| | | |
|---|---|---|
| Depression | Dorsocervical fat pad (“buffalo hump”) | Hypertension^{b} |
| Fatigue | Facial fullness | Incidental adrenal mass |
| Weight gain | Obesity | Vertebral osteoporosis^{b} |
| Back pain | Supraclavicular fullness | Polycystic ovary syndrome |
| Changes in appetite | Thin skin^{b} | Type 2 diabetes |
| Decreased | Peripheral edema | Hypokalemia |
| concentration | | |
| Decreased libido | Acne | Kidney stones |
| Impaired memory | Hirsutism or female balding | Unusual infections |
| (especially short term) | | |
| Insomnia | Poor skin healing | |
| Irritability | | |
| Menstrual | | |
| abnormalities | | |

^{a}Features are listed in random order.
^{b}CS is more likely if onset of the feature is at a younger age.
Reference:
Nieman, et al. The diagnosis of Cushing's syndrome: An Endocrine Society Clinical Practice Guideline. J Clin Endocrin Metab 2008; 98(5): 1526-1540, herein incorporated by reference.

VAS Questionnaire Criteria for Evaluating Physical Signs
of Cushing's Syndrome

| Subject + Physician | Physician Only | Subject Only |
|---|---|---|
| Facial plethora | Proximal myopathy | Easy bruising |
| | (or proximal muscle | |
| | weakness) | |
| Striae (especially if | | Hirsutism or female |
| reddish purple and >1 cm | | balding |
| wide) | | |
| Thin skin | | Poor skin healing |
| Dorsocervical fat pad | | Peripheral edema |
| (“buffalo hump”) | | |
| Facial fullness | | |
| Supraclavicular fullness | | |

The following physical signs of CS will be quantified by the Investigator using specific grading systems included in the SPM:
Acne (Doshi, et al. A comparison of current acne grading systems and proposal of a novel system. Int J Derm. 1997; 36: 416-418, herein incorporated by reference)
Hirsutism (Hatch, et al. Hirsutism: implications, etiology and management. Am J Obstet Gynecol. 1981; 140: 815-830, herein incorporated by reference)
Peripheral edema (Brodovicz, et al. Reliability and Feasibility of Methods to Quantitatively Assess Peripheral Edema. Clin Med Res 2009; 7(½): 21-31, herein incorporated by reference)

### Appendix D Conditions Associated with Pseudo-Cushing's Syndrome

Conditions Associated with Hypercortisolism in the Absence of Cushing's Syndrome
Reference: Nieman, et al. The diagnosis of Cushing's syndrome: An Endocrine Society Clinical Practice Guideline. J Clin Endocrin Metab 2008; 98(5): 1526-1540, herein incorporated by reference. 48 hour, 2 mg dexamethasone suppression test may be necessary to exclude pseudo-Cushing's disease. Normal cortisol ≤1.4 μg/dL.
   Some clinical features of CS may be present:
   Pregnancy
   Depression and other psychiatric conditions
   Alcohol dependence
   Glucocorticoid resistance
   Morbid obesity
   Poorly controlled diabetes mellitus
      Unlikely to have any clinical features of CS
   Physical stress (hospitalization, surgery, pain)
   Malnutrition, anorexia nervosa
   Intense chronic exercise
   Hypothalamic amenorrhea
   Cortisol-binding globulin (CBG) excess (increased serum but not urine cortisol)

### Appendix E Testing Criteria for Diagnosis of Diabetes Mellitus

The American Diabetes Association recommendations state that pre-diabetes (impaired glucose tolerance) and diabetes can be provisionally diagnosed with any one of the following criteria:

### Pre-Diabetes/Impaired Glucose Tolerance:

1. A fasting glucose of >100 and <126 mg/dL (after no caloric intake for at least 8 hours)

### Diabetes

1. A fasting glucose of ≥126 mg/dL (after no caloric intake for at least 8 hours) OR
2. A casual measurement of glucose ≥200 mg/dL (taken any time of day without regard to time of last meal) with classic diabetes symptoms: increased urination, increased thirst and unexplained weight loss OR
3. A glucose level on an oral glucose tolerance test (OGTT) (75 gram dose) of ≥200 mg/dL for the 2 hour sample. Oral glucose tolerance testing is not necessary if the subject has a glucose level of ≥126 mg/dL.

In the absence of unequivocal hyperglycemia with acute metabolic decompensation, the diagnosis should be confirmed on a subsequent day, by any of the above 3 criteria.

### 4.2.3 12-Lead Electrocardiograms (ECG)

Male Cushing's syndrome patients have been reported to have prolongation of the QT interval, which is not dissimilar to intervals reported in females. Thus, the inclusion criteria have allowed for this possibility and both males and females will be allowed into the study with baseline QTc≤470 msec.

12-Lead ECGs will be obtained during the study at baseline and at each dosing level within approximately 1 to 2 hours after drug administration (i.e., at approximately Cmax). ECGs will be obtained after the subject has rested in a supine position for at least 5 minutes, and should be conducted after the subject has fasted, or has not eaten within a minimum of 2 hours. (Food can prolong QTc intervals which may be incorrectly attributed to the 2S,4R ketoconazole enantiomer.) A small snack will be allowed for diabetic subjects and subjects who are intolerant of fasting, particularly on days when safety laboratory assessments (requiring an overnight fast) and ECGs are scheduled. The Spaulding Electrocardiograph device will be used to collect 5 minutes of continuous ECG at the times indicated in the Time and Events Table (Appendix A). The recording will be uploaded electronically to a computer and subsequently transmitted over the internet for centralized analysis and archiving. The system will provide automated measurements of HR, RR, PR, QRS, QT, QTcF and QTcB and ECG diagnostic statements to the investigative site within a short time following the completion and uploading of the recording. A central reader cardiologist, not involved in the conduct of the study, will subsequently overread the ECGs. It is imperative that the transmission of the ECGs to the central reader take place as soon as possible after the completion of the test to allow for the proper medical evaluation of the subject should QTc prolongations occur.

NOTE: ECGs at screening (single ECGs) are not required to be performed using the Spaulding Electrocardiograph device. The use of the Spaulding Electrocardiograph device will commence after the screening visit and must be used for all subsequent assessments.

The ECG analysis performed on the 5-minute ECGs will provide a summary ECG representative of the measurements and analysis of all normal beats within the 5-minute collection period, as well as an arrhythmia analysis based on the full recording. Variability indices will be calculated.

Subjects with prolonged QTc intervals >500 msec or >60 msec increase over baseline based on the 5-minute assessment using the Spaulding Electrocardiograph device must be medically observed until the QTc has returned to a value ≤500 msec. The subject should be evaluated for causality (as per recommendations in Section 6.2.9.3). The study medication may be withheld, after which subjects may resume study drug administration at lower doses of the drug once QTc interval is below 500 msec if the cause of the QTc prolongation is related to the 2S,4R ketoconazole enantiomer. Please see section 6.2.9.3 for special instructions to the Investigator on the assessment of QTc interval prolongations in this study. The Cortendo Medical Monitor should be contacted for guidance as needed.

### 4.2.4 Clinical Laboratory Tests

Samples for clinical laboratory testing will be collected at the times indicated in the Time and Events Table (Appendix A). A complete list of all the parameters, including an entire battery of LFTs, is provided in Appendix B. All routine testing will be conducted in a fasting state and in the morning. During the treatment phases, samples for laboratory evaluations will be collected during each dose level of the Dose Titration Phase and monthly during the Maintenance Phase. Additional laboratory parameters will include the following: serum cortisol, ACTH, coagulation parameters (prothrombin time [PT], partial thromboplastin time [PTT], International Normalized Ratio [INR]), triglycerides, serum lipid panel, HbAlc, CRP, serum free and total testosterone in both men and women, IGF-1 concentrations and spot urine for albumin/creatinine ratio to determine microalbuminuria.

TSH and free T4 will be measured at approximately 2 months after the start of dosing with the 2S,4R ketoconazole enantiomer during the titration phase. If subjects achieve their maximum 2S,4R ketoconazole enantiomer dose prior to 2 months during titration, their TSH and free T4 levels must be measured just prior to start of the maintenance phase (time=0).

Only data obtained from the central laboratory will be used for adjudication by the DSMB.

### 4.2.5 Dexamethasone Suppression Test (DST)

A DST will be administered if not previously performed within 2 months of the Screening visit to confirm eligibility for participation in the study, as well as at Month 12 in the Extended Evaluation phase. Available prior data (within 6 months) will be recorded in the CRF. In order to be fully interpretable, adherence to the timing requirement of the test will be important.

### Overnight Test

1-mg dose of dexamethasone will be administered orally between 11 PM and midnight
Blood sample to measure serum cortisol must be obtained at approximately 8 AM the next morning; the exact time to be recorded in the CRF
Nothing to drink or eat for at least 10 hours before the blood test

The following medications may affect the dexamethasone suppression test results and should be avoided as per exclusion criteria: barbiturates, corticosteroids, phenytoin, and tetracyclines.

Details of the assay are described in the SPM.

### 4.2.6 ACTH Levels

ACTH levels will be measured in all subjects at all study visits as listed in the Time and Events Table in Appendix A. Details of the assay are described in the SPM.

### 4.2.7 Oral Glucose Tolerance Test

All pre-diabetic subjects with screening fasting glucose concentrations >100 mg/dL but <126 mg/dL will have an oral glucose tolerance test (OGTT) at baseline and at the times indicated in the Time and Events Table (Appendix A). After an overnight fast of at least 12 hours, subjects will be asked to drink 75 g of glucose. Blood samples for the determination of glucose concentrations will be drawn before glucose administration and 30, 60, 90, and 120 minutes after administration. Subjects with diabetes, as defined in Appendix E, will be excluded from the OGTT.

### 4.2.8 Pituitary Magnetic Resonance Imaging (MRI)

Pituitary MRIs will be obtained to detect pituitary adenomas for subjects with Cushing's disease ONLY at baseline if not done within 6 months of administration of the first dose of the study drug, and at months 6 (for subjects with documented macroadenomas only) and month 12 (in all CD subjects). The MRI scans must be available for assessment prior to dosing. The results of MRIs obtained during the study will be evaluated by a central reader (neuroradiologist).

### 4.2.9 Special Considerations

### 4.2.9.1 Instructions for Subjects

Subjects will be instructed to carry a card at all times identifying the potential risk for adrenal insufficiency. This card will include subject's information, contact information for the Investigator, and the potential need for glucocorticoids in the settings of shock, surgery, and other conditions, as appropriate.

Subjects will be advised of the potential risk for adrenal insufficiency and be made aware of the signs and symptoms of this condition. At any time during the study, subjects should contact the clinical site in the event of any emerging clinical signs and symptoms of an AE. Clear instructions should be provided to the subject on how to access the medical staff at the investigational site regardless of day or time of day.

In addition, subjects will also be asked to carry an emergency kit containing hydrocortisone or another appropriate glucocorticoid, according to local medical practice, that can be administered immediately in case of adrenal insufficiency.

### 4.2.9.2 Considerations for Investigator

Subjects will be contacted (by subject preference) approximately 1 week after taking the first dose and approximately 1 week after each dose adjustment to check on subject status and ensure compliance with medication administration.

Throughout the study, subjects must be monitored and managed by the Investigator for the following diseases associated with CS or its treatment, according to recommended guidelines for diagnosis and standard of care:
Diabetes and impaired glucose tolerance: See Appendix for testing recommendations.
Hypertension: See Appendix C features of Cushing's syndrome
   Signs and symptoms of CS include the features listed in the table below; however, they should not be limited to the list. Additional signs and symptoms may be determined by the investigator, as appropriate.

Clinical Features of Cushing's Syndrome^{a}

| | | |
|---|---|---|
| Symptoms | Signs | Overlapping Conditions |

Features that best discriminate CS; most do not have a high sensitivity

Easy bruising
Facial plethora
Proximal myopathy (or
proximal muscle weakness)
Striae (especially if reddish
purple and >1 cm wide)

CS features in the general population that are common and/or less discriminatory

| | | |
|---|---|---|
| Depression | Dorsocervical fat pad | Hypertension^{b} |
| | (“buffalo hump”) | |
| Fatigue | Facial fullness | Incidental adrenal mass |
| Weight gain | Obesity | Vertebral osteoporosis^{b} |
| Back pain | Supraclavicular fullness | Polycystic ovary syndrome |
| Changes in appetite | Thin skin b | Type 2 diabetes |
| Decreased concentration | Peripheral edema | Hypokalemia |
| Decreased libido | Acne | Kidney stones |
| Impaired memory (especially | Hirsutism or female balding | Unusual infections |
| short term) | | |
| Insomnia | Poor skin healing | |
| Irritability | | |
| Menstrual abnormalities | | |

^{a}Features are listed in random order.
^{b}CS is more likely if onset of the feature is at a younger age.
Reference:
Nieman, et al. The diagnosis of Cushing's syndrome: An Endocrine Society Clinical Practice Guideline. J Clin Endocrin Metab 2008; 98(5): 1526-1540, herein incorporated by reference.

VAS Questionnaire Criteria for Evaluating Physical Signs
of Cushing's Syndrome

| Subject + Physician | Physician Only | Subject Only |
|---|---|---|
| Facial plethora | Proximal myopathy | Easy bruising |
| | (or proximal muscle | |
| | weakness) | |
| Striae (especially if | | Hirsutism or female |
| reddish purple and >1 cm | | balding |
| wide) | | |
| Thin skin | | Poor skin healing |
| Dorsocervical fat pad | | Peripheral edema |
| (“buffalo hump”) | | |
| Facial fullness | | |
| Supraclavicular fullness | | |

The following physical signs of CS will be quantified by the Investigator using specific grading systems included in the SPM:
Acne (Doshi, et al. A comparison of current acne grading systems and proposal of a novel system. Int J Derm. 1997; 36: 416-418, herein incorporated by reference)
Hirsutism (Hatch, et al. Hirsutism: implications, etiology and management. Am J Obstet Gynecol. 1981; 140: 815-830, herein incorporated by reference)
Peripheral edema (Brodovicz, et al. Reliability and Feasibility of Methods to Quantitatively Assess Peripheral Edema. Clin Med Res 2009; 7(½): 21-31, herein incorporated by reference)

### Appendix D Conditions Associated with Pseudo-Cushing's Syndrome

Conditions Associated with Hypercortisolism in the Absence of Cushing's Syndrome

Reference: Nieman, et al. The diagnosis of Cushing's syndrome: An Endocrine Society Clinical Practice Guideline. J Clin Endocrin Metab 2008; 98(5): 1526-1540, herein incorporated by reference. 48 hour, 2 mg dexamethasone suppression test may be necessary to exclude pseudo-Cushing's disease. Normal cortisol ≤1.4 μg/dL.

Some clinical features of CS may be present:
Pregnancy
Depression and other psychiatric conditions
Alcohol dependence
Glucocorticoid resistance
Morbid obesity
Poorly controlled diabetes mellitus
   Unlikely to have any clinical features of CS
Physical stress (hospitalization, surgery, pain)
Malnutrition, anorexia nervosa
Intense chronic exercise
Hypothalamic amenorrhea
Cortisol-binding globulin (CBG) excess (increased serum but not urine cortisol)

### Appendix E Testing Criteria for Diagnosis of Diabetes Mellitus

The American Diabetes Association recommendations state that pre-diabetes (impaired glucose tolerance) and diabetes can be provisionally diagnosed with any one of the following criteria:

### Pre-Diabetes/Impaired Glucose Tolerance:

2. A fasting glucose of >100 and <126 mg/dL (after no caloric intake for at least 8 hours)

### Diabetes

4. A fasting glucose of ≥126 mg/dL (after no caloric intake for at least 8 hours) OR
5. A casual measurement of glucose ≥200 mg/dL (taken any time of day without regard to time of last meal) with classic diabetes symptoms: increased urination, increased thirst and unexplained weight loss OR
6. A glucose level on an oral glucose tolerance test (OGTT) (75 gram dose) of ≥200 mg/dL for the 2 hour sample. Oral glucose tolerance testing is not necessary if the subject has a glucose level of ≥126 mg/dL.

In the absence of unequivocal hyperglycemia with acute metabolic decompensation, the diagnosis should be confirmed on a subsequent day, by any of the above 3 criteria.
Appendix for guidelines for classification of blood pressure levels and follow-up recommendations.
Hypocortisolemia: See Appendix for evaluations of signs and symptoms
Hypomineralcorticoidism: See Appendix for evaluations of signs and symptoms
Hypogonadism: See Appendix for evaluations of changes in sexual function as reported by the subject.
Adrenal insufficiency: All medical care providers identified by the subject, in addition to the Investigator, MUST be given sufficient written instructions about potential risks of adrenal insufficiency in subjects in this study.
   NOTE: It is well recognized that subjects with a good response to treatment for CS resulting in decreased, but not low, cortisol concentrations may exhibit signs and symptoms of cortisol withdrawal (non-life threatening) which may not be different than those are of adrenal insufficiency, which is a life threatening event warranting immediate medical intervention. Discrimination between the two may not be evident. Furthermore, subjects may have adrenal insufficiency without abnormal cortisol concentrations, but the concentrations, while within or above the normal range, are inappropriately low for the degree of physiological stress being experienced by the subject. These symptoms include: nausea, vomiting, abdominal pain, anorexia, malaise, fatigue, headache, arthralgias/myalgias, gastrointestinal discomfort, dizziness (particularly upon standing), irritability, depression, sweating, and fever. Subjects may have hypoglycemia as well. Therefore, subjects should be assessed for signs and symptoms AND cortisol levels as follows:
   Full review of symptoms
   lying and standing blood pressure measurements and pulse to evaluate postural changes
   temperature
   serum cortisol level; laboratory evidence of hypoadrenalism is defined as serum cortisol level <3 μg/dl (LLN or the lower limit of the assay being used for clinical testing) or a UFC measurement <LLN. Even if the serum cortisol and/or the UFC are within the normal range, the possibility of adrenal insufficiency should be considered, based on postural (orthostatic) vital sign changes and clinical signs and symptoms. Please see Appendix H for a list of clinical signs and symptoms.
   Should adrenal insufficiency be deemed present, the 2S,4R ketoconazole enantiomer should be temporarily discontinued for mild symptoms/signs.
   For moderate or severe symptoms/signs, the 2S,4R ketoconazole enantiomer will be temporarily discontinued and rescue glucocorticoids temporarily administered.
   The 2S,4R ketoconazole enantiomer at an appropriately lower dose can be restarted once the medical situation is deemed resolved. If the dose that caused adrenal insufficiency is 150 mg/day, the 2S,4R ketoconazole enantiomer should be completely discontinued, and the subject withdrawn from the study.

Proximal myopathy (or proximal muscle weakness) Easy bruising

### 4.2.9.3 Prolongation of QTc Interval >500 Msec or >60 Msec Above Baseline

The potential risk of protracted, drug induced prolongation of the QTc interval on the ECG is the development of an arrhythmia called torsade de pointes. The development of torsade de pointes in the face of QTc interval prolongation is rare, and appears to primarily occur when the QTc interval is particularly prolonged, generally >500 msec or >60 msec above baseline. Subjects with CS may have greater than normal QTc intervals and treatment that reduces cortisol levels may result in reductions in QTc intervals. The 2S,4R ketoconazole enantiomer appears to have the capability to prolong QTc intervals in a dose (concentration) related manner.

QTc interval is highly variable within a subject during the course of a day. It can also be prolonged by electrolyte abnormalities (low serum magnesium, calcium and potassium levels). Food intake can increase QTc intervals and necessitates that ECG evaluations be conducted in a fasting subject or that a minimum of 2 hours has elapsed since eating before the ECG is obtained. Nausea, vomiting, upset stomach, and dizziness can cause prolongation in the QTc interval as well.

Many drugs can prolong the QTc interval directly, or in the case of the 2S,4R ketoconazole enantiomer through interference with the metabolism of the 2S,4R ketoconazole enantiomer causing levels of the drug to increase, thus resulting in a prolongation of the QTc.

In this study, electrocardiographic monitoring, using the Spaulding Electrocardiographic device, is being performed at the designated times (as in the Time and Events table in Appendix A) continuously for 5 minutes. This 5-minute evaluation will provide an integrated summary ECG representative of the measurements and analysis of all heartbeats within the 5-minute collection period. It will be upon this integrated summary result that QTc prolongation will initially be identified. Unlike conventional ECG evaluation of the QTc which records three 10 second ECGs over 6-9 minutes, amounting to approximately 9-10 waveforms, the 5 min recording will provide a more robust evaluation of approximately 300 waveforms. The integrated summary of the ECG assessment (including arrhythmia analysis) will be done centrally after electronic transmission of the data, and the investigator will be provided with results in realtime. It is imperative that the Investigator or his/her designee downloads to the central reading site the Spaulding ECG shortly after collection to allow rapid assessment by the central reader and rapid results to the Investigator to ensure proper management of the subject.

With the exception of the Screening ECG, all ECGs must be evaluated using the Spaulding ECG device to ensure consistency in interpretation.

The following need to be considered in cases of QTc prolongation:
To emphasize all subjects should have fasted (minimum of 2 hours since food intake) prior to the performance of the scheduled ECG evaluation. If the integrated ECG reading demonstrates a QTc prolongation greater than 60 msec from baseline or an absolute QTc greater than 500 msec, the subject should be questioned about recent ingestion of food within the preceding 2 hours. If the subject has eaten, a repeat ECG evaluation should be conducted following a proper 2 hour fast. The subject should also be questioned about the use of any other medications that may have increased QTc interval, either directly because of their direct effect on the QTc interval or indirectly as a result of a drug interaction that may have increased the concentrations of the 2S,4R ketoconazole enantiomer. If such a drug is identified, it should be stopped immediately. Similarly, the subject should be evaluated for symptoms of nausea, recent vomiting prior to the ECG assessment, lightheadedness or other symptoms that may influence the QTc interval and potentially is driving the observed QTc interval changes. In this case, the ECG evaluation should be conducted once the subject's symptoms have abated. The 2S,4R ketoconazole enantiomer dosing may have to be withheld if the symptoms are protracted and the QTc interval prolongation remains evident. The Investigator should ensure that the symptoms as well as the ECG results are not due to a cardiovascular event, such as a myocardial infarction.
If the subject has not eaten, has no confounding medical symptoms/events, does not have a concomitant medication that may be increasing the QTc interval, and there are no other significant abnormalities on the ECG warranting immediate medical intervention, a repeat ECG evaluation should be undertaken within approximately 30 minutes of the observed ECG prolongation or as soon as it is practical.
If the repeat ECG evaluation continues to demonstrate QTc prolongation beyond 60 msecs above baseline or an absolute value of greater than 500 msec, a blood sample should be obtained from the subject for evaluation of electrolyte abnormalities, including magnesium and calcium concentrations in addition to routine electrolytes, including potassium, and for a PK evaluation. If electrolyte abnormalities are identified, these should be corrected before re-evaluation of the ECG. The 2S,4R ketoconazole enantiomer may be temporarily withheld until electrolytes can be normalized.
If no other cause can be identified to account for the absolute QTc interval >500 msec (or >60 msec above baseline), and the ECG evaluation on repeat determination continues to demonstrate QTc prolongation, this would be defined as persistent elevation of the QTc interval due to the 2S,4R ketoconazole enantiomer, and the 2S,4R ketoconazole enantiomer should be withheld. The ECG should be intermittently monitored until resolution of the QTc prolongation. The 2S,4R ketoconazole enantiomer at a lower dose can be restarted after a washout period of 2 days. ECG evaluation should be continued. In all cases, abnormal morphology of the ECG (especially T wave changes), if present, will be recorded in the CRF. The Investigator may request consultation from a cardiologist should there be any uncertainty in the management of the subject if deemed necessary.
If the drug is discontinued, repeat ECG evaluation and PK sampling of the 2S,4R ketoconazole enantiomer will be undertaken during the washout of the drug.

### 4.2.10 Pregnancy

Regardless of post-menopausal status, all female participants must have a negative pregnancy test at screening and at study visits indicated in the Time and Events Table (Appendix A). A urine βhCG will be performed at the screening visit and all other subsequent visits. Results of the test must be available before dosing can begin.

Post-menopausal females will be defined as having amenorrhea for a minimum of 24 consecutive months and an elevated FSH level at baseline.

Reports of pregnancies in female subjects (or in female partners of male subjects) will be collected after the start of dosing and until 5 terminal half-lives following the last dose. Female subjects found to be pregnant will be withdrawn from the study.

### 4.2.10.1 Action to be Taken if Pregnancy Occurs

The Investigator will collect pregnancy information on any female subject or partner of a male subject who becomes pregnant while participating in this study. The Investigator will record pregnancy information on the appropriate form and submit it to the Sponsor within 24 hours of learning of the pregnancy. The subject will also be followed to determine the outcome of the pregnancy. Generally, follow-up will be no longer than 6 to 8 weeks following the estimated delivery date. Any premature termination of the pregnancy will be reported.

All pregnancies will be reported from the site and documented according to the same procedures as SAE. While a pregnancy will not be considered as an SAE, any pregnancy complication or elective termination of a pregnancy for medical reasons will be recorded as an AE or SAE.

A spontaneous abortion is always considered to be an SAE and will be reported as such. Furthermore, any SAE occurring as a result of a post-study pregnancy and which is considered reasonably related to the investigational product by the Investigator, will be reported to the Sponsor. While the Investigator is not obligated to actively seek this information in former study participants, he or she may learn of an SAE through spontaneous reporting. Subjects will be asked to contact the clinic in these situations.

### 4.3 Efficacy Assessments

### 4.3.1 Urinary Sample Collections

Urine will be collected in the provided containers. Subjects will be asked to provide either 2 or 4 adequate 24-hour urine collections (may be sequential) at the times indicated in the Time and Events Table (Appendix A). Subjects must also follow the dietary restrictions listed in Section 5.

At baseline, subjects will be asked to provide 4 adequate 24-hour urine collections. It is recommended that the 4 urine samples be collected on sequential days and brought to the clinic within 1-2 days of the last collection since UFC is only stable for 7 days under refrigerated storage conditions.

After the start of each dose and in order to assess the need for further dose titration, subjects will be asked to collect 2 adequately collected 24-hour urine specimens starting at approximately Day 10 (+2 days). They will bring their 2 urine collections to the clinic on approximately Day 12 (+2 days). Urine volume will be measured at the clinic and samples will then be shipped frozen to the designated laboratory for measurement of UFC levels from each collection.

24-hour urine collections will be obtained monthly during the maintenance phase and at 9 and 12 months during the extended evaluation phase. Four adequate 24-hour urine collections will be obtained at the completion of the titration phase, prior to entry into the maintenance phase to confirm a therapeutic dose has been reached, at months 3 and 6 months of the maintenance phase. These urine collections are absolutely critical to the interpretation of the study results and the subject should be made fully aware of the need for their compliance with this collection. At all other monthly visits during the maintenance phase and at months 9 and 12 of the extended evaluation phase, 2 adequate 24-hour collections will be obtained.

Urine collections will begin after the first morning void (Day 1) and will be collected over 24 hours until the next morning, including the first void on the morning of Day 2. Details of the assay are described in the SPM. Careful instructions must be provided to the subject on the process for proper urine collection and storage of samples over 24 hours.

The total volume of urine and urine creatinine excretion rates will also be measured from the 24-hour collections as a marker of the adequacy of the collection. Total urine volume should be 400 to 4000 mL/day. Collections judged to be inadequate may be repeated at discretion of the investigator.

Guidelines for 24-hour creatinine excretion rates are provided in Table 2. It is important to note that in healthy male volunteers within the confines of a clinical research unit, a considerable day-to-day variability in urinary creatinine excretion has been reported with standard deviations of 10.5% to 14.4% ranging from 50% to 79% of the mean. In subjects that have not been confined, the day-to-day variability in the 24-hour urinary creatinine excretion rates has been reported to be approximately 46%. More recent data in Cushing's disease subjects demonstrated intrasubject variability in UFC was approximately 50%, and increased as UFC levels increased with intrasubject variability in corresponding urine creatinine measurements of approximately 33%.

TABLE 2
Normal 24-Hour Creatinine Excretion from Adequate Urine Collections

| | |
|---|---|
| Males | 1. Age 20 to 50: 18.5 to 25.0 mg/kg/day |
| | 2. Age 50 to 70: 15.7 to 20.2 mg/kg/day |
| | 3. Range: 0.8 to 1.8 grams per 24 hours |
| Females | 1. Age 20 to 50: 16.5 to 22.4 mg/kg/day |
| | 2. Age 50 to 70: 11.8 to 16.1 mg/kg/day |
| | 3. Range: 0.6 to 1.6 grams per 24 hours |

Due to the muscle wasting observed in CS and dependent on the length of the disease, subjects may have 24-hr urine creatinine excretion rates in the lower end of the ranges noted above. Subjects should be instructed to avoid consuming ≥4 L/day of liquids on the day of urine collection (see also Section 5).

### 4.3.2 Urinary Sample Analysis

UFC levels from the 24-hour urine collection will be assayed at a central laboratory according to the current validated methodology. Details for handling and reporting of 24-hour UFC measurements and urine creatinine excretion rates will be provided in the SPM.

### 4.3.3 Late Night Salivary Cortisol

Late night salivary cortisol samples will be collected at the times indicated in the Time and Events Table (Appendix A). Saliva collections must be done at between 11 pm and midnight and following the dietary restrictions listed in Section 5. Three late night samples (one sample for each of 3 nights), will be collected at baseline and at 3 time points during the Maintenance phase. Single late night samples (one night only) will be collected at Months 1, 2, 4 and 5 of the Maintenance phase and at all other scheduled visits. All samples will be analyzed at a central laboratory with experience in salivary cortisol measurements. All details for collection and handling of samples are provided in the SPM.

### 4.4 Pharmacokinetics

### 4.4.1 Pharmacokinetic Sample Collection

Blood samples (1 mL minimum) for the determination of plasma concentrations of the 2S,4R ketoconazole enantiomer will be collected at the following nominal times as outlined below (Table 3):

TABLE 3
Pharmacokinetic Sampling Scheme

| Planned | |
|---|---|
| Sequence | Nominal Time |
| Sequence 1 | Predose (0), 2-4 hours post-dose, 24 hours post-dose |
| Sequence 2 | Predose (0) |
| Sequence 3 | Predose (0), and 0.5-1 hour post-dose |
| Sequence 4 | Predose (0) |
| Sequence 5 | Predose (0) |
| Sequence 6 | Predose (0) and 2-4 hours post-dose |
| Sequence 7 | Predose (0), and 0.5-1 hour post-dose, and 4-6 hours |
| | post-dose |

The above PK sampling scheme will begin at the titration phase and will continue sequentially until all PK samples have been collected. If a subject completes all dose levels in the dose titration, then the Sequence 1-7 samples would be collected at each dose level for DLs 1-7, respectively. In cases when the PK sampling scheme has not been completed during the titration phase (i.e., a subject has not completed all 7 dose levels during the dose titration phase), the sampling scheme will be continued in the Maintenance Phase until all PK samples at the indicated nominal times have been collected. At DL1, if a subject's dose is decreased from BID dosing to once daily, the PK sampling scheme is to be followed as per the planned schedule above.

The actual PK times may vary from the nominal times; however, the actual time of sample collection as well as the time of dose administration and the actual dose administered prior to the PK sample collection MUST be recorded. See SPM for further details.

### 4.4.2 Pharmacokinetic Sample Analysis

Sample analysis will be performed by Alturas Analytics (Moscow, Indiana USA) under the direction of Cortendo AB. Concentrations of the 2S,4R ketoconazole enantiomer will be determined using current methodology.

### 4.5 Quality of Life Measures

The Cushing QoL questionnaire will be utilized for measurement of quality of life at the times indicated in the Time and Events Table (Appendix A).

### 5, Lifestyle and Dietary Restrictions

Subjects must follow the following lifestyle and dietary restrictions for study-specific assessments.
Consumption of grapefruit and blood orange products should be avoided throughout the study.
During the 24-hour urine collections, subjects must refrain from the following:
   Drinking ≥4 L/day of fluids
   Use of medicines or products with glucocorticoids, such as hemorrhoid or skin creams that contain steroids
On the days that samples are taken for salivary cortisol test, subjects must refrain from the following within 1 hour of the test:
   Eating genuine licorice
   Smoking cigarettes
   Chewing tobacco
Information on subject's job related to working shift hours will be collected.

### 6. Investigational Products

### 6.1 Description of Investigational Product

The 2S,4R ketoconazole enantiomer will be provided as 150 mg tablets. The tablets will be ⅜″ round, biconvex, and unmarked with a pink film coat and supplied in foil induction sealed HDPE bottles.

### 6.2 Dosage and Administration

The 2S,4R ketoconazole enantiomer will be administered BID, approximately every 12 hours, according to a titration scheme described in Section 3.1 approximately every 13 to 17 days. Subjects will receive an adequate number of tablets at each dose titration visit prepared by the study pharmacist.

After dose level 1, at the discretion of the Investigator, subjects may be dosed on a TID schedule (approximately every 8 hours) if the Investigator believes the subject will benefit from this dosing interval; however, the total daily dose at each dose level, as indicated in Table 1, should not be altered.

### 6.3 Dose Rationale

Treatment of CS with any therapeutic agent has not been predictable. Ketoconazole, the most commonly used off-label therapeutic agent with the best therapeutic success rate (reported as 50-70%) of drugs used to treat CS, has variable PK, and is titrated to effect, even rarely up to levels as high as 1800 mg to control the severely ill CS patients. In most patients responsive to ketoconazole, doses average to approximately 600-800 mg/day with the range of 200-1800 mg having been utilized to achieve control.

Given that the 2S,4R ketoconazole enantiomer is the more potent enantiomer of ketoconazole in vitro and the severity of the disease manifestations of CS, starting with a dose of the 2S,4R ketoconazole enantiomer that is expected to have some medical benefit seems most appropriate. Like ketoconazole, and unlike some drugs used to treat CS, the 2S,4R ketoconazole enantiomer is not expected to be administered at a fixed dose, but will be titrated to effect in each subject. Please see full discussion on the rationale for dose selection, based on the characteristics of the 2S,4R ketoconazole enantiomer in comparison to racemic ketoconazole, in the Investigator's Brochure, herein incorporated by reference.

At common clinical doses, based on the observed half-life of the 2S,4R ketoconazole enantiomer, a BID dosing scheme would be appropriate for optimal peak-to-trough concentration ratios in most, but not all subjects. In this study, 150 mg of COR 003 administered BID has been chosen as the starting dose in the titration schema with upwards dosing in increments of 150 mg daily. However, it should be noted that some subjects may benefit from TID dosing, particularly when higher doses are required. This option will be allowed in the study, at the discretion of the Investigator, as long as the total daily dose at each dose level is not exceeded.

The highest dose of 600 mg BID of the 2S,4R ketoconazole enantiomer (1200 mg total daily dose) is allowed during this trial, because some CS subjects may require higher levels of cortisol suppression to achieve control in this severe disease. As with other doses, titration to this level will be guided by tolerability and safety parameters. It is expected that titration to this highest dose will be the exception rather than the norm, based on the experience with ketoconazole. Unlike some other drugs used for CS, the direct inhibition of cortisol production by the 2S,4R ketoconazole enantiomer allows for explicit measurement of cortisol levels in the urine to ensure the minimum amount of drug is used to achieve therapeutic efficacy, and thus it will ensure that excessive drug administration does not occur.

Understanding the characteristics of dose and exposure responses of the 2S,4R ketoconazole enantiomer in a highly controlled trial with investigator oversight will allow for an informative evaluation of its risks and benefits prior to use in medical practice and will better serve to inform the management of the subject.

### 6.4 Blinding

This will be an open-label study.

### 6.5 Randomization and Treatment Assignment

This is a non-randomized study. All subjects that meet eligibility criteria and sign informed consent will begin treatment on 150 mg BID, approximately every 12 hours.

### 7. Drug Supplies, Dispensing, Storage and Accounting

### 7.1 Product Accountability

The Investigator is responsible for investigational product accountability, reconciliation, and record maintenance. In accordance with all applicable regulatory requirements, the Investigator or designated site staff must maintain investigational product accountability records throughout the course of the study. The responsible person(s) will document the amount of investigational product received from and returned to Cortendo (when applicable), the amount supplied and/or administered to and returned by subjects, if applicable.

### 7.2 Compliance Assessment

Subjects will be asked to complete diary cards to record medication administration and contacted (by subject preference) during the study to ensure compliance with medication administration. In addition, at all visits, subjects will be questioned regarding compliance and a pill count will be obtained. Compliance will be further confirmed by measuring drug concentrations throughout the study.

### 7.3 Treatment of Investigational Product Overdose

There is no known antidote to the 2S,4R ketoconazole enantiomer. Subjects should be medically managed according to their clinical condition, as appropriate. Known potential risk to an overdose of the 2S,4R ketoconazole enantiomer may be prolongation of the QTc interval.

### 8. Concomitant Medications

### 8.1 Permitted Medications

Over-the-counter liquid and tablet antacids are allowed, but must be used in moderation and taken ≥2 hours after dosing with the 2S,4R ketoconazole enantiomer. A list will be provided.

### 8.2 Prohibited Medications

During the study, subjects are not allowed to take the following medications. The investigator is encouraged to contact the Cortendo Medical Monitor for any questions.
Megesterol acetate
Total daily dose of acetaminophen >3 g, due to increased hepatotoxicity
Prescription or over the counter H2 receptor antagonists, proton-pump inhibitors or sucralfate (which inhibit absorption of the drug), except over-the-counter antacids as described above in Section 8.1.
Weight loss medications (prescription or over-the-counter)
Medications that are strong CYP3A4 inhibitors or CYP3A4 inducers as they may interfere with the metabolism of the 2S,4R ketoconazole enantiomer. In particular, rifampicin, rifabutin, isoniazid, nevirapine and phenytoin will significantly reduce the 2S,4R ketoconazole enantiomer concentrations. In particular, ritonavir will increase the 2S,4R ketoconazole enantiomer concentrations. A list of other medications with these effects is provided in Appendix G, and while they are not absolutely prohibited, they should be avoided if alternative therapy is available.
Statins other than pravastatin, fluvastatin and rosuvastatin
Common Medications resulting in QTc prolongation as a direct effect or as a result of interaction with the 2S,4R ketoconazole enantiomer: domperidone, alfuzosin, clarithromycin, astemizole, bepridil, halofantrine, disopyramine, cisapride, dofetilide, levacetylmethadol (levomethadyl), mizolastine, moxifloxacin pimozide, quinidine, sertindole or terfenadine. Investigator should confer with sponsor as necessary.
The following herbal medicines: St John's Wort, echinacea, gingko, goldenseal, yohimbe, red rice yeast, danshen, silybum marianum, Asian ginseng, schissandra sphenanther, shankhapushi, and Asian herb mixture (Xiao chai hu tang and Salboku-to)
Topical steroids
Inhaled steroid medications
Carbamazepine (assay interference), fenofibrate (assay interference), carbenoxolone
Excessive ingestion of genuine licorice (has mineralocorticoid effects)

Subjects on treatment for CS for whom treatment has been inadequate or not well tolerated will have to agree to a washout period as determined by the nature of their treatment before baseline assessments are performed for participation in this study:
Inhibitors of steroidogenesis (including ketoconazole): 2 weeks
Dopamine agonists: bromocriptine (2 week), cabergoline (8 weeks)
Octreotide acetate LAR and lanreotide Autogel®: 12 weeks
Lanreotide SR/long-acting pasireotide: 8 weeks
Octreotide acetate (immediate release formulation) or short-acting pasireotide: 1 week
Mifepristone (RU 486): 4 weeks
Mitotane: >6 months and have a serum concentration <5 μg/mL
   8.3 Medications to be Used with Caution
Medications that have a large first pass metabolism largely mediated by CYP3A4 and a narrow therapeutic margin: Alprazolam, midazolam, triazolam, nisoldipine, lovastatin, simvastatin, eplerenone, ergot alkaloids (ergotamine, dihydroergotamine), and sirolimus. The 2S,4R ketoconazole enantiomer may affect the metabolism of other drugs listed in Appendix G but they are NOT necessarily prohibited. However, due to the potential for increased exposure by the 2S,4R ketoconazole enantiomer, careful monitoring, with possible adjustment in dosage, is recommended for the following drugs: alfentanil, fentanyl, sulfentanil, HIV drugs (indinavir, squinavir), calcium channel blocking drugs (amlodipine, felodipine, nicardipine, nifedipine), bosentan, buspirone, sildanfil, busulfna, cilostazol, digoxin, docetaxel, paclitaxel, oral anti-coagulants, immunosuppressants (cyclosporine, tacrolimus, rapamycin), niconazole telithromycin, tolterdodine, trimetrexate, verapramil, vinca alkaloids (vincristine, vinblastine, vinorelbine)

### 9. Subject Completion and Withdrawal

### 9.1 Subject Completion

Subjects who have completed all protocol-specified assessments in the maintenance phase of the study will be considered to have completed the study.

### 9,2 Subject Withdrawal Criteria

Refer to Section 3.1 for dose titration, modification and stopping criteria based on safety criteria.

Subjects will be withdrawn if they experience a serious or intolerable AE that prevents them from completing the study.

A subject may withdraw from study treatment at any time at his/her own request, or may be withdrawn at any time at the discretion of the Investigator for safety, behavioral or administrative reasons.

### 9.3 Withdrawal Procedures

Reasons for withdrawal (subjects who refuse to return for any remaining study visits) or discontinuation (subjects who prematurely stop the treatment) at any time during the study may include, but are not limited, to the following:
For safety reasons, either at the discretion of the Investigator or at the subject's request
For protocol violations at the discretion of the Sponsor
Due to concomitant therapy that could interfere with the results of the study, (the Investigator will report all such information on the CRFs and decide, in accordance with the Sponsor, whether the subject is to be withdrawn).

All premature discontinuations and their causes must be carefully documented by the Investigator on the CRF, and, if need be, on the AE form.

If, for any reason, a subject is withdrawn before completing the Maintenance or Extended Phases of the study, the reason for termination will be recorded. All data gathered on the subject prior to termination will be made available to the Sponsor. Subjects not completing the entire study should be fully evaluated when possible.

If the subject chooses to withdraw before completing the study, the subject should return to the clinic to complete all safety and PK assessments as outlined for the Early Termination (E/T) visit in Appendix A.

### 10. Data Analysis and Statistical Considerations

### 10.1 Population for Analysis

Intent-to-Treat (ITT) Population: The ITT population will include all subjects who receive at least one dose of the 2S,4R ketoconazole enantiomer. This population will be used for the evaluation of efficacy and all safety analyses.

Per Protocol (PP) Population: The PP population will consist of all subjects who enter the maintenance phase of the study, receive at least 80% of the planned doses and have no major protocol deviations that may affect efficacy. This population will be used for evaluation of efficacy, if different from the ITT population.

### 10.2 Hypothesis

The primary endpoint for this study will be response to the 2S,4R ketoconazole enantiomer, defined as mean UFC concentration ≤ULN following 6 months of dosing in the maintenance phase without a prior dose increase during that phase. The proportion of responders in the ITT and PP populations will be estimated along with corresponding 95% confidence intervals (CIs). If the lower bound of the 95% CI in the ITT population is greater than or equal to 20% in the ITT population then the 2S,4R ketoconazole enantiomer will be considered effective.

### 10.3 Sample Size Determination

A sufficient number of subjects (estimated at approximately 90) will be enrolled into the dose titration phase of the study to ensure that at least 70 subjects complete the 6 month assessment period in the maintenance phase of the study. Assuming that the response rate in the ITT population is 35% and 90 subjects are enrolled in the dose titration phase, the half-width of the 95% CI will be within approximately 10% of the estimated response rate.

In addition, the sample size of 90 subjects will provide half-widths of confidence intervals around the mean changes from baseline for the following secondary endpoints:

| | | Estimated |
|---|---|---|
| | | Precision of 95% |
| | Assumed | CIs following |
| | Standard | administration |
| | Deviation in | of the 2S,4R |
| | Change from | ketoconazole |
| | Baseline | enantiomer |
| Fasting Glucose (mmol/L) | 1.6 | 0.33 |
| HbAl C (%) | 0.75 | 0.15 |
| Systolic Blood Pressure (mmHg) | 21.1 | 4.36 |
| Diastolic Blood Pressure (mmHg) | 14.9 | 3.08 |
| Triglycerides (mg/dL) | 0.97 | 0.20 |
| LDL (mg/dL) | 1.29 | 0.27 |
| Body Weight (kg) | 5.9 | 1.22 |

As an example, if the observed mean change from baseline to visit in fasting glucose is −2.0 mmol/L with a standard deviation of 1.6 mmol/L in 90 subjects, then the estimated 95% confidence interval around the mean change from baseline in fasting serum glucose will be −2.33 to −1.67.

### 10.4 General Considerations for Data Analysis

The SAS System, Version 9.1.3 (or higher), will be used for all analyses, unless otherwise specified. Continuous variables (e.g., age) will be summarized using the number of observations (n), mean, standard deviation (SD), median, minimum, and maximum. Categorical variables (e.g., sex, race) will be summarized using the number of observations (n) and percentage in each category. All data used in analyses and/or collected during the study will be provided in listings.

### 10.4.1 Premature Withdrawal and Missing Data

Subjects who withdraw prior to the Month 6 assessment in the maintenance phase will be considered as prematurely withdrawn. All available data for subjects who prematurely withdraw from the study will be included in all analyses. During every period in which four 24 hour UFC collections are conducted, the dataset will be considered complete if at least two out of the four collections have been adequately collected.

Analysis of data for specific visits will be performed using two different approaches to account for missing data:
1. RLOCF (Restricted Last Observation Carried Forward) analysis: This involves bringing (carrying) forward the last non-missing post-baseline assessment following Month 3 for subjects with missing data and/or for subjects who discontinue from the study. Subjects who withdraw prior to the Month 4 assessment will be considered treatment non-responders in the primary analysis and their data will not be carried forward.
2. At Visit analysis: Missing values at post-baseline assessments will not be replaced and will be regarded as missing.

Analyses will be performed as appropriate using the RLOCF as the primary approach along with the At Visit approach as supportive, if appropriate. Details will be provided in the statistical analysis plan.

### 10.5 Final Analyses

### 10.5.1 Safety Analyses

### 10.5.1.1 Extent of Exposure

Study drug exposure will be summarized as the average daily dose, cumulative dose, and total number of days on study drug. Total number of days on study drug will be calculated for each subject as the treatment stop date minus treatment start date plus one day.

Study drug compliance between visits and cumulative study drug compliance will be calculated by dividing the number of study drug tablets used (total number dispensed minus total number returned/lost/wasted) by the total number of study drug tablets prescribed and multiplying the result by 100.

### 10.5.1.2 Adverse Events (AEs)

AEs will be coded using MedDRA and graded according to the National Cancer Institute Common Terminology Criteria for Adverse Events, version 3.0.

The proportion of subjects reporting at least one AE, at least one drug-related AE, at least one serious AE, at least one AE leading to discontinuation of study drug, and at least one AE leading to withdrawal from the study will be computed. The number and the percentage of subjects reporting each AE will be summarized for all AEs, and separately for drug-related AEs, all serious AEs, all AEs leading to discontinuation of study drug, and all AEs leading to withdrawal from the study.

The most common AEs are defined as those occurring in at least 5% of the ITT population. The number and the percentage of subjects reporting the most common AEs will be summarized.

### 10.5.1.3 Clinical Laboratory Evaluations

A laboratory value that is within the normal range will be considered normal. A laboratory value that is outside the testing laboratory's normal range will be considered an abnormal laboratory value. The number and percentage of subjects with abnormal laboratory values will be summarized for each scheduled visit.

Differences in laboratory values between baseline and each scheduled laboratory assessment will be calculated for each laboratory test and summarized descriptively. The laboratory test values obtained just prior to the start of treatment will be used as the baseline value. Further details of the categorical analysis of the laboratory evaluation will be defined in detail in the statistical analysis plan.

### 10.5.1.4 Vital Signs

Heart rate and three measurements of SBP and DBP will be taken at each visit. The mean of these measurements will be used as the value for each visit. Baseline will be defined as the mean of the values from the pre-dose measurement at baseline. Change from baseline for each parameter will be summarized. In addition, values of clinical importance (Table 4) will be identified in the data listings.

TABLE 4
Vital Sign Values of Clinical Importance

| Vital Sign | Criteria | | Flag |
|---|---|---|---|
| Heart Rate | <44 | bpm | Low (L) |
| | 44-100 | bpm | Normal |
| | 101-120 | bpm | High (H) |
| | >120 | bpm | Very High (VH) |
| Systolic Blood Pressure | <90 | mmHg | Low (L) |
| | 90-139 | mmHg | Normal |
| | 140-169 | mmHg | High (H) |
| | ≥170 | mmHg | Very High (VH) |
| Diastolic Blood Pressure | <50 | mmHg | Low (L) |
| | 50-89 | mmHg | Normal |
| | 90-109 | mm H19 | High (H) |
| | ≥110 | mmHg | Very High (VH) |

The number and percentage of subjects with blood pressure and heart rate outside the pre-defined range of clinical concern at any post-baseline assessment will be summarized.

Ambulatory blood pressure monitoring (ABPM) assessments including change from baseline will be listed and summarized by study day and time point. Mean 24-hour ABPM measurements and awake and sleep time mean ABPM measurements will be included in the summary table. Mean 24 hour ABPM will be assessed with an ANOVA model with study day as a fixed effect. Mean differences in 24 hour mean ABPM and associated 95% CIs between each post-baseline assessment and baseline will be estimated to determine overall change from baseline in ABPM following dosing of the 2S,4R ketoconazole enantiomer. Additional analyses may be performed, if warranted.

### 10.5.1.5 Electrocardiograms (ECGs)

Summary ECGs, based on the measurement of an ECG over 5 minutes as described in Section 4.2.3 will be used for each evaluation. The number and percentage of subjects with any clinically significant abnormal change in the ECG evaluations will be summarized. ECG parameters (PR interval, QRS duration, HR, QT interval, QTcB interval, and QTcF interval) and changes from baseline will be summarized descriptively. ECGs results will be available to the Investigator shortly after being obtained and all ECGs will be reviewed by a central consulting cardiologist. These data will be used in the evaluation of the drug effect.

### 10.5.1.6 Physical Examination Findings

Physical examination findings will be listed by subject and visit and summarized as appropriate. At baseline and at 6 and 12 months, the adjudication committee, composed of three clinician-investigator experts in CS will assess the subjects for signs of hypercortisolism (facial rubor, supraclavicular and dorsal fat pads, and general overall body habitus) by scoring standardized photographs on a scale from 0 to 3 (with 0 indicating no signs, 1 mild hypercortisolism, 2 moderate hypercortisolism, and 3 severe hypercortisolism).

### 10.5.1.7 Other Safety Measures

Results from the DST at screening and 12 months will be listed by subject. ACTH levels will be listed and summarized descriptively by timepoint. Results from pituitary MRI will be listed by subject and timepoint. IGF-1 concentrations will be listed by subject and summarized descriptively by timepoint.

### 10.5.2 Efficacy Analyses

All efficacy analyses will be performed on the ITT and PP populations using the RLOCF approach. RLOCF will be applied for subjects that were prematurely withdrawn from the study.

Withdrawn subjects prior to the Month 4 assessment and those who reach the maximum tolerated dose without a response will be considered non-responders.

### 10.5.2.1 Primary Efficacy

The proportion of responders at 6 months in the maintenance phase in the ITT and PP populations will be estimated along with corresponding 95% CIs. The 95% CI for the proportion of responders will be calculated based on the normal approximation to the binomial distribution. If the lower bound of the 95% CI in the ITT population is ≥20% in the ITT population, then the 2S,4R ketoconazole enantiomer will be considered effective.

### 10.5.2.2 Secondary Efficacy

### UFC Analysis

The proportion of responders to the 2S,4R ketoconazole enantiomer (defined similarly to the primary endpoint) along with associated 95% CIs will be estimated at 1, 2, 3, 4 and 5 months of dosing in the maintenance phase and at 9 and 12 months in the extended evaluation phase.

Recognizing the stringency of the definition of UFC for the primary endpoint, the proportion of subjects with mean UFC≤ULN or ≥50% reduction from baseline at month 6 of the maintenance phase of dosing will be calculated and reported as well. This proportion of responders to the 2S,4R ketoconazole enantiomer along with the associated 95% CI will be estimated.

Mean UFC will be measured from 24-hour urine collections. The mean UFC from the collections at each visit will be used in the analysis of UFC. UFC will be summarized by timepoint using descriptive statistics. In addition, change from baseline and percent change from baseline will be calculated for each post-baseline assessment. Mean and individual plots of each endpoint over time will be presented.

In addition, a shift table will be created to summarize the shift from baseline to each post-baseline timepoint using the following UFC normality categories: less than LLN, normal range, greater than ULN and less than 2×ULN, 2× to 5×ULN, 5×ULN to 10×ULN, greater than 10×ULN.

### Salivary and Serum Cortisol Levels

Mean salivary and serum cortisol levels at 1, 2, 3, 4, 5 and 6 months of dosing in the maintenance phase and at 9 and 12 months in the extended evaluation phase including change from baseline will be listed and summarized descriptively by assessment time.

### Biochemical Markers of CS Comorbidities

Biochemical markers of CS comorbidities (lipid profile to include total HDL/LDL cholesterol, lipid ratios and triglycerides, fasting serum glucose levels, HbAlc, oral glucose tolerance tests CRP, spot albumin/creatinine ratios (as a measure of microalbuminuria), ACTH, IGF-1 and testosterone concentrations (females only) at designated times during the maintenance phase and at 9 and 12 months during the extended evaluation phase including change from baseline will be listed and summarized descriptively by assessment time. In addition, shifts from baseline (based on laboratory normal ranges) will be summarized by number and percent of subjects in each category.

### Clinical Signs and Symptoms

Specifically targeted clinical signs and symptoms, defined in Appendix C, will be summarized at each timepoint by number and percent of subjects for qualitative measurements and descriptive statistics for quantitative measurements. In addition, shift tables in clinical signs and symptoms by assessment time will be created to demonstrate any improvement gained during the course of treatment.

### Quality of Life Questionnaire

Quality of life measures and changes from baseline will be summarized by timepoint using descriptive statistics.

### Medication Changes

Changes to concomitant medications as a result of exposure to COR-003 will be listed by subject. In addition, the number and percentage of subjects with medication changes during the course of the study will be summarized. Correlations between changes in medication will be made with clinical and laboratory data as appropriate. Special and specific emphasis will be placed on collecting the details of the medication changes made for blood pressure, diabetes and cholesterol management.

### 10.5.2.3 Exploratory Efficacy

Post-dose assessments of secondary endpoints (including clinical signs, symptoms and laboratory parameters) may be statistically compared with baseline assessments to determine statistically significant improvements, if appropriate and data permit. Further details will be provided in the statistical analysis plan.

In addition, primary and secondary endpoints may be descriptively compared with a similar population analyzed in a clinical trial of pasireotide as described. No formal statistical comparisons will be made. Further details will be provided in the SAP.

### 10.5.3 Pharmacokinetic Analysis

PK model parameters will include: clearance (CL/F), volume of distribution (V/F), absorption rate constant (Ka) with associated between subject variability where feasible. Model parameters will be tabulated with associated precision. Derived parameters including half-life (t1/2), area under the concentration time curve (AUC) and peak concentration (Cmax) will be reported, if appropriate. Because the PK of the 2S,4R ketoconazole enantiomer are reported to change over time, time dependent changes in CL/F AUC and t1/2 will be investigated and if identified, the changes will be described. Derived parameters will be tabulated with associated summary statistics.

Pharmacokinetic data will be evaluated using a population modeling based approach as implemented in NONMEM® (Version 7 level 2 or higher). Subjects with at least one adequately documented dose and concentration record will be considered for inclusion in the population pharmacokinetic evaluation. All evaluations will be conducted based on a pre-specified analysis plan. Standard model building and model evaluation procedures will be followed. Derived parameters will be calculated from the final model.

### 10.5.4 Pharmacodynamic Analysis

The PD model parameters including maximal suppression of UFC (Imax), the 2S,4R ketoconazole enantiomer dose producing half maximal UFC suppression (IC50) and associated estimates of between subject variability will be reported. Individual maximal UFC reduction achieved will be tabulated and summary statistics (mean, median, standard deviation and percent coefficient of variation) will be presented. Stochastic simulations of the expected response for the preferred dose regimen will be generated to explore the range of UFC reduction.

PD data (UFC) will be evaluated using a population modeling based approach as implemented in NONMEM® (Version 7 level 2 or higher). Subjects included in the population analysis with at least one adequately documented UFC record will be considered for inclusion in the population PD evaluation. All evaluations will be conducted based on a pre-specified analysis plan. Standard model building and model evaluation procedures will be followed. Derived parameters such as maximal response will be calculated from the final model as appropriate.

### 10.5.5 Subgroup Analyses

### 10.5.5.1 Prior Therapy for Cushing's Syndrome

Subgroup efficacy analyses may be performed for subjects that enter the study as treatment naive and for subjects on prior therapy or failing prior therapy.

### 10.5.5.2 Prior Radiation Therapy

Subgroup efficacy analyses will be performed excluding subjects who previously received radiation therapy. The total number of previously irradiated subjects will not exceed 10. If the UFC levels for these subjects, with prior radiation therapy, do not increase at the follow-up visit after a decrease has been observed while on the 2S,4R ketoconazole enantiomer therapy, it will not be possible to determine if the decrease in UFC levels while on the 2S,4R ketoconazole enantiomer could be due to the 2S,4R ketoconazole enantiomer therapy or previous radiation, which is known to have a delayed response.

### 10.5.5.3 Hypertensive Subjects

Subgroup efficacy analyses will be performed for subjects who enter the study and are prescribed anti-hypertensive medications or subjects who enter the study with a baseline systolic blood pressure >130 mmHg or diastolic blood pressure ≥90 mmHg.

### 10.5.5.4 Pre-Diabetic/Diabetic Subjects

Subgroup efficacy analyses will be performed for subjects who enter the study as pre-diabetic (baseline fasting serum glucose >100 mg/dL but <126 mg/dL) or diabetic (baseline fasting serum glucose ≥126 mg/dL).

### 11. Adverse Events (AE) and Serious Adverse Events (Sae)

The Investigator and study staff are responsible for detecting and recording AEs and SAEs, during scheduled safety evaluations and whenever such information is brought to their attention. This section of the protocol provides definitions and detailed procedures to be followed.

During each visit, the Investigator will question the subject about adverse events using an open question taking care not to influence the subject's answers, e.g. “have you noticed any change in your health?”

### 11.1 Definition of an AE

An AE is any untoward medical occurrence in a study subject which is temporally associated with the use of a medicinal product, regardless of its potential relationship to the medicinal product. An AE, therefore, can be any unfavorable or unintended sign, including an abnormal laboratory finding, symptom, or disease (new or exacerbated), whether or not related to the study drug.

Examples of an AE include:
Exacerbation of a pre-existing condition including either an increase in frequency and/or intensity of the condition.
New conditions detected or diagnosed after investigational product administration even though it may have been present prior to the start of the study.
Signs, symptoms of a drug interaction
Signs, symptoms of a suspected overdose of either investigational product or a concurrent medication
A laboratory abnormality occurring after the start of the study (i.e., after screening) that results in subject withdrawal from the study or medical treatment or further follow-up.

NOTE: Abnormal laboratory values at the screening visit that preclude a subject from entering the study are not considered adverse events, but will be recorded.

AEs may include pre- or post-treatment events that occur as a result of protocol-mandated procedures (i.e., invasive procedures, modification of subject's previous therapeutic regimen).

### 11.2 Definition of a SAE

A serious adverse event is any untoward medical occurrence that, at any dose:
(a) results in death.
(b) is life-threatening.

NOTE: The term ‘life-threatening’ in the definition of ‘serious’ refers to an event in which the subject was at risk of death at the time of the event. It does not refer to an event, which hypothetically might have caused death, if it were more severe.
(c) requires hospitalization or prolongation of existing hospitalization.

NOTE: In general, hospitalization signifies that the subject has been detained (usually involving at least an overnight stay) at the hospital or emergency ward for observation and/or treatment that would not have been appropriate in the physician's office or out-patient setting. Complications that occur during hospitalization are AEs. If a complication prolongs hospitalization or fulfills any other serious criteria, the event is serious. When in doubt as to whether “hospitalization” occurred or was necessary, the AE should be considered serious.
(d) results in disability/incapacity, or

NOTE: The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g. sprained ankle) which may interfere or prevent everyday life functions but do not constitute a substantial disruption.
(e) is a congenital anomaly/birth defect.
(f) Medical or scientific judgment should be exercised in deciding whether reporting is appropriate in other situations, such as important medical events that may not be immediately life-threatening or result in death or hospitalization but may jeopardize the subject or may require medical or surgical intervention to prevent one of the other outcomes listed in the above definition. These should also be considered serious. Examples of such events are invasive or malignant cancers, intensive treatment in an emergency room or at home for allergic bronchospasm, blood dyscrasias or convulsions that do not result in hospitalization, or development of drug dependency or drug abuse.

### 11.2.1 Disease-Related Events

Although symptoms of CS are quite non-specific, all AEs and their attributions will be collected and reviewed. The reduction in cortisol levels, regardless of therapeutic intervention, is known to cause symptoms (e.g., nausea, lethargy, muscle aches), and these too will be captured as AEs for evaluation.

### 11.2.2 Symptom-Specific Assessment of AEs

During dose titration, at predose to every dose escalation visit and at each subsequent study visit, in addition to spontaneous AE reporting, symptom-specific AEs will be assessed, which will include, but not be limited to, the evaluation of the following: fatigue/malaise, nausea, vomiting, headache, anorexia, myalgias, arthralgias, dizziness, pruritus, right upper quadrant pain, abdominal pain/discomfort, fever, dark or acholic stools. Instructions on capturing these AEs will be provided in the SPM.

### 11.3 Time Period, Frequency, and Method of Detecting AEs and SAEs

As a consistent method of soliciting AEs, the subject should be asked a non-leading question such as: “How do you feel?”

All AEs occurring after obtaining the informed consent until the end of the final visit must be reported. All AEs must be recorded irrespective of whether they are considered drug-related.

At each visit/assessment in the period defined above, AEs will be evaluated by the Investigator and recorded.

Any AEs already documented at a previous assessment and designated as ongoing, should be reviewed at subsequent visits as necessary. If these have resolved, this should be documented. Changes in intensity or frequency of AEs should be recorded as separate events (i.e., a new record started).

The recording of AEs and SAEs are described in Section 11.4 (“Recording of AEs and SAEs”).

### 11.4 Recording of AEs and SAEs

All clinical events, including either observed or volunteered problems, complaints or symptoms are to be recorded on the Adverse Events page(s) of the CRF. The need to capture this information is not dependent upon whether the clinical event is associated with study treatment. Adverse clinical events resulting from concurrent illnesses or reactions to concurrent medications are also to be recorded. In order to avoid vague, ambiguous, or colloquial expressions, the AE should be recorded in standard medical terminology rather than the subject's own words.

Each adverse clinical event is to be evaluated for duration, intensity, and whether the event may be associated with the study drug or other causes. Start and stop dates, relationship to study drug, medical management, and alternative causality of event must be recorded in the Adverse Events section of the CRF. AEs believed to be possibly related to study drug must be followed until resolution.

### 11.5 Evaluating AEs and SAEs

### 11.5.1 Severity Rating

The severity of an adverse event (AE and SAE) will be graded according to National Cancer Institute Common Terminology Criteria for Adverse Events, Version 3.0.

| | |
|---|---|
| Grade 1 | Mild; asymptomatic or mild symptoms; clinical or diagnostic |
| | observations only; intervention not indicated |
| Grade 2 | Moderate; minimal, local or noninvasive intervention indicated; |
| | limiting age-appropriate instrumental activities of daily living |
| Grade 3 | Severe or medically significant but not immediately life- |
| | threatening; hospitalization or prolongation of hospitalization |
| | indicated; disabling; limiting self care activities of daily living |
| Grade 4 | Life-threatening consequences; urgent intervention indicated. |
| Grade 5 | Death related to AE. |

An AE that is assessed as severe should not be confused with a SAE. An event is defined as ‘serious’ when it meets one of the pre-defined outcomes as described in Section

### 11.2 “Definition of a SAE”.

### 11.5.2 Relationship to Study Drug

SAEs will be classified as “not related” or “related” (including unknown).

For AEs, the relationship to study treatment is to be assessed according to the following definitions:

Definitely not related: The AE is definitely not related to the drug. This designation should be reserved for those events which occur prior to study treatment or for those events which cannot be even remotely related to study participation (e.g., injuries sustained in an automobile accident).

Unlikely: There is no reasonable association between the study treatment and the suspected event and the event could have been produced by the subject's clinical state or other modes of therapy administered to the subject.

Possibly related: The suspected adverse event may or may not follow a reasonable temporal sequence from study treatment administration but seems to be the type of reaction that cannot be dismissed as unlikely. The event could have been produced or mimicked by the subject's clinical state or by other modes of therapy concomitantly administered to the subject.

Probable: The suspected adverse event follows a reasonable temporal sequence from study treatment administration, abates upon discontinuation of the treatment, and cannot be reasonably explained by the known characteristics of the subject's clinical state.

Definitely related: This designation should be reserved for those events which have no uncertainty in their relationship to treatment administration.

### 11.6 Follow-Up of AEs and SAEs

After the initial AE/SAE report, the Investigator is required to proactively follow each subject. Further information on SAEs will be provided to the Sponsor's designated safety group on the subject's condition within 24 hours.

New or updated information will also be recorded on the “SAE” CRF within 24 hours.

### 12. Additional References the Following References are Each Incorporated Herein by Reference

Agarwal R. Reproducibility of renal function measurements in adult men with diabetic nephropathy: research and clinical implications. Am J Nephrol 2007; 27(1):92-100.
American Diabetes Association: Standards of medical care in diabetes (Position Statement). Diabetes 2005; 28:S4-S36.
Bochicchio D, Losa M, Buchfelder M. Factors influencing the immediate and late outcome of Cushing's disease treated by transsphenoidal surgery: a retrospective study by the European Cushing's Disease Survey Group. J Clin Endocrinol Metab. 1995 November; 80(11):3114-20.
Brodovicz, KG, McNaughtan K, Uemura N, Meininger G, Girman C J, Yale S H. Reliability and Feasibility of Methods to Quantitatively Assess Peripheral Edema. Clin Med Res 2009; 7(½):21-31
Castinetti F, Guignat L, Giraud P, et al. Ketoconazole in Cushing's disease: is it worth a try? J Clin Endocrinol Metab. 2014; 99:1623-1630.
Colao A, Petersenn S, Newell-Price J, Findling J, Gu F, Maldonado M, Schoenherr U, Mills D, Salgado R, and Biller B for the Pasireotide B2305 Study Group. A 12-Month Phase 3 Study of Pasireotide in Cushing's Disease. N Engl J Med 2012; 366, 914-924.
Dalton G. Transsphenoidal hypophysectomy for pituitary tumours. Proc R Soc Med. 1974 September; 67(9):885-9.
Daneshmend T K, Warnock D W. Clinical Pharmacokinetics of Ketoconazole. Clinical Pharmacokinetics. 1988; 14:13-34.
De Martin M, Pecori Giraldi F, Cavagnini F. Cushing's disease. Pituitary. 2006; 9(4):279-87.
Doshi A, Zaheer A, Stiller M J. A comparison of current acne grading systems and proposal of a novel system. Int J Derm. 1997; 36:416-418
Engelhardt D, Weber M M, Miksch T, Abedinpour F, Jaspers C. The influence of ketoconazole on human adrenal steroidogenesis: incubation studies with tissue slices. Clinical Endocrinology. 1991; August; 35(2):163-8.
Fleseriu M, Biller B M K, Findling J, Molitch M, Schteingart D, Gross C on behalf of the SEISMIC Study Investigators. Mifepristone, a Glucocorticoid Receptor Antagonist, Produces Clinical and Metabolic Benefits in Patients with Cushing's Syndrome. J Clin Endocrinol Metab 2012; 97: 2039-2049.
Gerber J D, Dilmaghania S, Gal J. Stereoselective pharmacokinetics (PK) of oral ketoconazole (K) in healthy subjects. Interscience Conference on Antimicrobial Agents and Chemotherapy (ICAAC) September 2003; poster #A-1569.
Giraldi F P, Toja P M, Michailidis G, Metinidou A, De Martin M, Scacchi M, Stramba-Badiale M, Cavagnini F. High Prevalence of Prolonged QT Interval Duration in Male Patients with Cushing's Disease. Exp Clin Endocrinol Diabetes 2011; 119:221-224.
Greenblatt D J, Ransil B J, Harmatz J S, Smith T W, Duhme D W, Koch-Weser J. Variability of 24-hour urinary creatinine excretion by normal subjects. J Clin Pharmacol 1976; 16(7):321-8.
Hatch R, Rosenfield R L, Kim M H, Tredway D. Hirsutism: implications, etiology, and management. Am J Obstet Gynecol. 1981; 140:815-830.
Kreutzer J, Jeske I, Hofmann B, Blumcke I, Fahlbusch R, Buchfelder M, et al. No effect of the PPAR-gamma agonist rosiglitazone on ACTH or cortisol secretion in Nelson's syndrome and Cushing's disease in vitro and in vivo. Clin Neuropathol. 2009 November-December; 28(6):430-9.
Kucers A, Hoy J F, Lindsay Grayson M, Crowe S M. The Use of Antibiotics: A Clinical Review of Antibacterial, antifungal and antiviral drugs. In: Butterworth, Hindman, editors. 5 ed: Oxford University Press; 1997. p. 1358-84.
Lake-Bakaar G, Scheuer P J, Sherlock S. Hepatic reactions associated with ketoconazole in the United Kingdom. Br Med J. 1987; 294:419-422.
Lewis J H, Zimmerman H J, Benson G D, Ishak K G. Hepatic injury associated with ketoconazole therapy. Analysis of 33 cases. Gastroenterology. 1984; 86:503-513.
Lindholm J, Juul S, Jorgensen J O, Astrup J, Bjerre P, Feldt-Rasmussen U, et al. Incidence and late prognosis of cushing's syndrome: a population-based study. J Clin Endocrinol Metab. 2001 January; 86(1):117-23.
Mancini T, Kola B, Mantero F, Boscaro M, Arnaldi G. High cardiovascular risk in patients with Cushing's syndrome according to 1999 WHO/ISH guidelines. Clinical endocrinology. 2004 December; 61(6):768-77.
National Cancer Institute. Common Terminology Criteria for Adverse Events (NCI CTCAE), version 4.0. Jun. 14, 2014 http://ctep.cancer.gov/protocolDevelopment/
Nieman L K, Biller B M K, Findling J W, et al. The diagnosis of Cushing's syndrome: an Endocrine Society Clinical Practice Guideline. J. Clin Endocrinol & Met. 2008; 93(5):1526-1540.
Nieman L K. Medical therapy of Cushing's disease. Pituitary. 2002; 5(2):77-82.
Nizoral® Product Insert. Janssen Pharmaceuticals. July 2013.
Petersenn S, Newell-Price J, Finding J W, Gus F, Maldonado M, Sen K, Salgado L R, Colao A, and Biller B M K on behalf of the Pasireotide B2305 Study Group. High variability in baseline urinary free cortisol values in patients with Cushing's disease. Clin Endocrinol. 2013; 0: 1-9.
Pivonello R, Faggiano A, Lombardi G, Colao A. The metabolic syndrome and cardiovascular risk in Cushing's syndrome. Endocrinol Metab Clin North Am. 2005 June; 34(2):327-39, viii.
Pozza C, Graziadio C, Giannetta E, Lenzi A, Isiori A. Management strategies for aggressive Cushing's syndrome: from macroadenomas to ectopics. J Oncol. 2012; 2012, Article ID 685213, 9 pages.
Rodriguez R J, Acosta D, Jr. N-deacetyl ketoconazole-induced hepatotoxicity in a primary culture system of rat hepatocytes. Toxicology. 1997 Feb. 28; 117(2-3):123-31.
Rotstein D M, Kertesz D J, Walker K A, Swinney D C. Stereoisomers of ketoconazole: preparation and biological activity. J Med Chem. 1992 Jul. 24; 35(15):2818-25.
Schwartz S L, Rendell M, Ahmann A J, Thomas A, Arauz-Pacheco C J, Welles B R. Safety profile and metabolic effects of 14 days of treatment with DIO-902: results of a phase IIa multicenter, randomized, double-blind, placebo-controlled, parallel-group trial in patients with type 2 diabetes mellitus. Clin Ther. 2008 June; 30(6): 1081-8.
Sharma S T, Nieman L K. Cushing's syndrome: all variants, detection, and treatment. Endocrinol Metab Clin North Am. 2011 June; 40(2):379-91, viii-ix.
Sonino N. The use of ketoconazole as an inhibitor of steroid production. N Engl J Med. 1987; 317:812-8.
Steen R E, Kapelrud H, Haug E, Frey H. In vivo and in vitro inhibition by ketoconazole of ACTH secretion from a human thymic carcinoid tumour. Acta endocrinologica. 1991 September; 125(3):331-4.
Steffensen C, Bak A M, Rubeck K Z, Jorgensen J O. Epidemiology of Cushing's syndrome. Neuroendocrinology, 2010; 92: Suppl 1: 1-5
The Seventh Report of the Joint National Committee on Prevention, Detection, Evaluation, and Treatment of High Blood Pressure (JNC 7)—Complete Report. August 2004.
Webb S M, Badia X, Barahona M J, et al. Evaluation of health-related quality of life in patients with Cushing's syndrome with a new questionnaire. Eur J Endocrinol 2008; 158; 623-30.
Welles B. Glucocorticoids in type 2 Diabetes Mellitus and the Metabolic Syndrome. Drug Dev Res 2006; 67:570-3.
Whitehouse L W, Menzies A, Mueller R, Pontefract R. Ketoconazole-induced hepatic phosphplipidosis in the mouse and its association with de-N-acetyl ketoconazole. Toxicology. 1994; 94:81-95.

### LIST OF ABBREVIATIONS AND DEFINITION OF TERMS

ACTH Adrenocorticotropic hormone

### AE Adverse Event

### AP Alkaline Phosphatase

ALT Alanine aminotransferase
ABPM Ambulatory blood pressure monitoring
AST Aspartate aminotransferase
AUC Area under the concentration time curve
BID Twice daily
BIPSS Bilateral inferior petrosal sinus sampling
BMI Body mass index
BhCG Beta human chorionic gonadotropin
CBG Cortisol-binding globulin

### CD Cushing's Disease

### CI Confidence Interval

CL/F Apparent clearance following oral administration
Cmax Peak concentration
CRH Corticotropin-releasing hormone
CRP C-reactive protein
CS Cushing's svndrome
DBP Diastolic blood pressure
DIM Drug-induced liver injury

### DL Dose Level

### DSMB Data Safety Monitoring Board

### DST Dexamethasone Suppression Test

### ECG Electrocardiogram

F/r Early termination
eGFR Estimated glomerular filtration rate

### FDA Food and Drug Administration

FSH Follicle stimulating hormone
GFR Glomerular filtration rate
GGT Gamma-glutamyl transferase

### HbAlc Hemoglobin A 1C

HDL High density lipoprotein

### HIPAA Health Insurance Portability and Accountability Act of 1996

MR Heart rate
IC50 Half maximal suppression

### ICH International Conference on Harmonization

### IEC Independent Ethics Committee

### IGF-1 Insulin-like Growth Factor-1

Imax Maximal suppression of UFC

### INR International Normalized Ratio

### IRB Institutional Review Board

### ITT Intent-to-Treat

IUD Intrauterine device
Ka Absorption rate constant
LDL Low density lipoprotein
LFT Liver function test
LLN Lower limit of normal
LOCF Last observation carried forward

### MDRD Modified Diet in Renal Disease

### MRI Magnetic Resonance Imaging

NASH Non-alcoholic steatohepatitis

### NCI-CTCAEv3 National Cancer Institute Common Terminology for Adverse Events, Version 3.0

OGTT Oral glucose tolerance test

### OST Overnight Suppression Test

### PK Pharmacokinetics

### PP Per Protocol

QD Once daily
QoL Quality of life
RLOCF Restricted last observation carried forward

### SAE Serious Adverse Event

SBP Systolic blood pressure

### SPM Study Procedures Manual

t½ Half-life

### T2DM Type 2 Diabetes Mellitus

TSH Thyroid stimulating hormone
UFC Urinary free cortisol
ULN Upper limit of normal
V/F Apparent volume of distribution following oral administration
VAS Visual analog scale
WNL Within normal limits
WOCP Women of child-bearing potential

### Annendix A Time and Events Tables

| | Screening and Baseline: Perform | | | | | | | | | |
|---|---|---|---|---|---|---|---|---|---|---|
| | Informed Consent PRIOR to ANY | | | | | | | | | |
| | Study Related Procedures and | | Dose Titration | | | | | | | |
| | Complete the Following: ^{1, 2} | | DL1 to | | | | | | | |
| | Initial | | therapeutic | | | | | Extended Evaluation | | |
| | Screening | Baseline | dose or | Maintenance Phase | | | | Phase | | |
| Assessments | Procedures | Procedures | DL7 | Month 1⁵ | Months 2, 4, 5 | Months 3, 6 | Early Termination³ | Month 9 | Month 12 | Follow Up⁴ |
| Check | X | X (within | | | | | | | | |
| Inclusion/Exclusion | | 15 days | | | | | | | | |
| criteria | | prior to | | | | | | | | |
| | | first dose) | | | | | | | | |
| Medical History and | X | X (within | | | | | | | | |
| demography⁶ | | 30 days | | | | | | | | |
| | | prior to | | | | | | | | |
| | | first dose) | | | | | | | | |
| Record prior/review | X | X (within | X | X | X | X | X | X | X | X |
| concomitant | | 30 days | | | | | | | | |
| medication⁷ | | prior to | | | | | | | | |
| | | first dose) | | | | | | | | |
| Admin/Review drug | | | X ^{8, 9} | X (Day 1, M | X | X | | X | X | |
| dispensing log/pill | | | | I only) | | | | | | |
| count | | | | | | | | | | |
| Physical examination | X | X (within | X | X | X | X | X | X | X | X |
| including assessment of | | 15 days | | | | | | | | |
| appearance¹⁰ | | prior to | | | | | | | | |
| | | first dose) | | | | | | | | |
| Vital signs (blood | X | X (within | X | X | X | X | X | X | X | X |
| pressure & heart rate)¹¹ | | 30 days | | | | | | | | |
| | | prior to | | | | | | | | |
| | | first dose) | | | | | | | | |
| Ambulatory blood | | X (within | | X (M0 only) | | X | | | | |
| pressure | | 30 days | | | | | | | | |
| | | prior to | | | | | | | | |
| | | first dose) | | | | | | | | |
| Weight and height/ | X | X (within | X | X | X | X | X | X | X | X |
| BMI/body habitus | | 30 days | | | | | | | | |
| | | prior to | | | | | | | | |
| | | first dose) | | | | | | | | |
| Abdominal Girth¹²12 | | X (within | | X (M0 only) | | X | X | X | X | X |
| | | 30 days | | | | | | | | |
| | | prior to | | | | | | | | |
| | | first dose) | | | | | | | | |
| HIV/Hepatitis B and | X | | | | | | | | | |
| C blood test | | | | | | | | | | |
| Standardized | | X (within | | | | X (Month 6 only) | | | X | |
| photographs | | 30 days | | | | | | | | |
| | | prior to | | | | | | | | |
| | | first dose) | | | | | | | | |
| ECGs¹³ - Fasting | X (using | X (Spaulding | X | X (M0 only) | X | X | X | X | X | X |
| | site ECG) | remainder | | | | | | | | |
| | | of trial) | | | | | | | | |
| | | (within 15 | | | | | | | | |
| | | days prior | | | | | | | | |
| | | to first | | | | | | | | |
| | | dose) | | | | | | | | |
| Contact subject (by | | | X | | | | | | | |
| subject preference) to | | | | | | | | | | |
| check on subject status | | | | | | | | | | |
| and ensure compliance | | | | | | | | | | |
| with medication | | | | | | | | | | |
| administration. | | | | | | | | | | |
| Late night salivary | | X (3 | | See | X (One night) | X (3 nights) | X (One night) | X (One night) | X (One night) | X (One night) |
| cortisol test | | nights) | | Assessment | | | | | | |
| | | (within 30 | | Schedule for | | | | | | |
| | | days prior | | X (3 nights | | | | | | |
| | | to first | | for M0 | | | | | | |
| | | dose) | | assessment) | | | | | | |
| | | | | (One night | | | | | | |
| | | | | for M2 | | | | | | |
| | | | | assessment) | | | | | | |
| TSH measurement/ | X | | X¹⁴ (at 2 | X (M0 only | X (M2 & 4 only) | X (M6 only) | | X | X | |
| free T4 | | | months) | if not done | | | | | | |
| | | | | during Dose | | | | | | |
| | | | | Titration) | | | | | | |
| Pituitary MRI for | | X (if not | | | | X (Macroade | X | | X | X (If not |
| Cushing's disease only | | done within | | | | nornas | | | | done at M |
| | | 6 months | | | | only) (M 6 | | | | 12 visit) |
| | | of first | | | | only) | | | | |
| | | dose) | | | | | | | | |
| FSH - females only | X | | | | | | | | | |
| Urine βHCG (women | X | X (within | X | X | X | X | X | X | X | X |
| only) | | 15 days | | | | | | | | |
| | | prior to | | | | | | | | |
| | | first dose) | | | | | | | | |
| Safety clinical | X | X (within | X | X | X | X | X | X | X | X |
| chemistry (including | | 15 days | | | | | | | | |
| LFTs), hematology, | | prior to | | | | | | | | |
| urinalysis - fasting ¹⁵ | | first dose) | | | | | | | | |
| INR/PT/PTT | | X (within | | X | X | X | X | X | X | X |
| | | 15 days | | | | | | | | |
| | | prior to | | | | | | | | |
| | | first dose) | | | | | | | | |
| HbA1c Measurements | X | X (within | | X (M0 only) | | X | X | X | X | X |
| | | 15 days | | | | | | | | |
| | | prior to | | | | | | | | |
| | | first dose) | | | | | | | | |
| OGTT (pre-diabetics | | X (within | | X (M0 only) | | X | X | | | |
| only) | | 15 days | | | | | | | | |
| | | prior to | | | | | | | | |
| | | first dose) | | | | | | | | |
| Mitotane Level: (>6 | X | | | | | | | | | |
| months since last dose) | | | | | | | | | | |
| (if necessary) | | | | | | | | | | |
| Dexamethasone | | X (within 2 | | | | | | | X | |
| suppression test | | months | | | | | | | | |
| | | prior to | | | | | | | | |
| | | first dose) | | | | | | | | |
| Serum cortisol¹⁶ | | X (within | X | X | X | X | X | X | X | X |
| | | 30 days | | | | | | | | |
| | | prior to | | | | | | | | |
| | | first dose) | | | | | | | | |
| ACTH (all subjects) | | X (within | X | X | X | X | | X | X | |
| | | 15 days | | | | | | | | |
| | | prior to | | | | | | | | |
| | | first dose) | | | | | | | | |
| Serum lipid | | X (within | X | X | X | X | X | X | X | X |
| measurements, | | 15 days | | | | | | | | |
| triglycerides | | prior to | | | | | | | | |
| | | first dose) | | | | | | | | |
| CRP measurements | | X (within | | | | X | X | X | X | X |
| | | 15 days | | | | | | | | |
| | | prior to | | | | | | | | |
| | | first dose) | | | | | | | | |
| IGF-1 levels | | X (within | | X | X | X | X | X | X | X |
| | | 15 days | | | | | | | | |
| | | prior to | | | | | | | | |
| | | first dose) | | | | | | | | |
| Microalbuminuria | | X (within | | X (M0 only | | X (If | X (If | X (If | X (If | |
| assessment (spot urine | | 15 days | | if abnormal | | abnormal at | abnormal at | abnormal | abnormal at | |
| assessing albumin/ | | prior to | | at baseline) | | baseline) | baseline) | at | baseline) | |
| creatinine ratio) | | first dose) | | | | | | baseline) | | |
| Serum testosterone | | X (within | X | X | X | X | X | X | X | X |
| measurements, | | 15 days | | | | | | | | |
| free/total (males and | | prior to | | | | | | | | |
| females) | | first dose) | | | | | | | | |
| Baseline signs & | X | X (within | X | X | X | X | X | X | X | X |
| symptoms/AEs/ | | 30 days | | | | | | | | |
| symptom- specific AEs | | prior to | | | | | | | | |
| (collect throughout | | first dose) | | | | | | | | |
| study) | | | | | | | | | | |
| CushingQoL | | X (within | | | | X | X | X | X | X |
| questionnaire | | 15 days | | | | | | | | |
| | | prior to | | | | | | | | |
| | | first dose) | | | | | | | | |
| 24-hour urinary free | | X (4 | X (2 | X (2 | X (2 | X (4 | X (4 | X (2 | X (2 | X (2 |
| cortisol/creatinine | | collections) | collections) | collections | collections) | collections) | collections) | collections) | collections) | collections |
| measurement/urinary | | (within 30 | ¹⁸ When | for M1 | | | | | | |
| volume¹⁷ | | days prior | UFC | assessment) | | | | | | |
| | | to first | results | | | | | | | |
| | | dose) | from first 2 | | | | | | | |
| | | | samples ≤ | | | | | | | |
| | | | ULN | | | | | | | |
| | | | request 2 | | | | | | | |
| | | | additional | | | | | | | |
| | | | collections | | | | | | | |
| | | | to confirm | | | | | | | |
| | | | therapeutic | | | | | | | |
| | | | dose has | | | | | | | |
| | | | been | | | | | | | |
| | | | reached. | | | | | | | |
| Pharmacokinetic | | | X ^{19,20} (M0 only) | X (only | X (only | X (only | | | | |
| sampling | | | | required for | required for | required for | | | | |
| | | | | subjects who | subjects | subjects | | | | |
| | | | | did not | who did | who did | | | | |
| | | | | complete all | not | not | | | | |
| | | | | 7 dose levels | complete | complete | | | | |
| | | | | in dose | all 7 dose | all 7 dose | | | | |
| | | | | titration | levels in | levels in | | | | |
| | | | | period. | dose | dose | | | | |
| | | | | Continue | titration | titration | | | | |
| | | | | until all 7 | period. | period. | | | | |
| | | | | collection | Continue | Continue | | | | |
| | | | | sequences | until all 7 | until all 7 | | | | |
| | | | | have been | collection | collection | | | | |
| | | | | collected) | sequences | sequences | | | | |
| | | | | | have been | have been | | | | |
| | | | | | collected) | collected) | | | | |

¹ Initial Screening Procedures: Perform these procedures immediately following ICF for all subjects (prior to washout if washout is necessary)
² Baseline Procedures: Perform these procedures following ICF and following washout (if washout is necessary) ONLY if initial screening procedure was performed outside of the defined window (see below) or procedure was not required at initial screening.
³Early Termination: All subjects that stop participation prior to completing Month 6 of maintenance will be considered as Early Termination and the following procedures are required within 2 weeks of last treatment. For subjects previously treated by radiation, the follow-up visit will be >2 but ≤4 weeks of last treatment.
⁴All subjects that complete Month 6 of maintenance, regardless of completion of EE Phase will require Follow Up within 2 weeks of last treatment. For subjects previously treated by radiation, the follow-up visit will be >2 but ≤4 weeks of last treatment.
⁵Month 1: (Includes Month 0 (M0) and Month 1 (M1) Assessments. Refer to Assessment Schedule for timing.
⁶Medical history to include previous documentation of diagnosis of Cushing's syndrome, and data on all previous LFTs, as medical records permit. Medical histories should be updated at Baseline.
⁷Blood pressure, diabetes, cholesterol medications will be specifically reviewed
⁸ Subjects will be contacted within approximately 1 week after taking their first dose of the study medication and after each new dose level during titration
⁹ Subjects reaching dose levels of >600 mg/day of total daily dose, will be dosed at the clinic and will be asked to return 4-7 days and again at 14-16 days after the dose escalation for safety evaluations.
¹⁰As part of the physical examination assessments, subjects and the study physician will also complete questionnaires or grading scales/grading instruments related to the physical signs of CS. The “Targeted Signs and Symptoms of Clinical Interest Form” questionnaire and VAS questionnaires are completed at screening, baseline, once per dose level, each month during the Maintenance phase, months 9 and 12 during the Extended phase and on early termination or follow-up visit. For VAS questionnaires (subject and study physician) repeat assessment at 4-7 days and 14-16 after dose escalation for dose levels >600 mg/day. See Study Procedures Manual.
¹¹Blood pressure will be measured in triplicate after resting for 10 minutes. Repeat assessment at 4-7 days and 14-16 after dose escalation for dose levels >600 mg/day
¹²Abdominal girth will be measured in triplicate. See the SPM for instructions on measuring abdominal circumference
¹³ECGs (over 5 minutes) will be obtained using a Spaulding Electrocardiograph device taken at baseline, and at each dosing level within approximately 1 to 2 hours after drug administration at each dose level (i.e., at~Cmax), and monthly at the therapeutic dose during the maintenance phase until month 6. Only screening ECGs may be conducted using available ECG machines.
¹⁴TSH/free T4 to be measured after 2 months of dosing during the titration phase. If, subjects reach their maximum dose before 2 months during the titration phase, then they will have TSH/free T4 measured at time 0 of the maintenance phase.
¹⁵ See Appendix B, Laboratory Parameters, includes LFTs. Repeat assessment at 4-7 days and 14-16 after dose escalation for dose levels ≥600 mg/day.
¹⁶Repeat serum cortisol and symptom-specific assessment of AEs at 4-7 days and 14-16 after dose escalation for dose levels ≥600 mg/day.
¹⁷Previously irradiated subjects, must stop treatment at the end of the maintenance phase for ≥2 weeks and have 4 complete 24-hour urine collections for UFC measurements. Subjects may resume dosing after the urine collections at the discretion of the investigator.
¹⁸ First urine 24-hour urine collection will be ~Day 10 (±2 days) and the second collection will be ~Day 11 (±2 days) after start of each dose level. Collections may be sequential.
¹⁹ Sequence 1 predose (0), 2-4 hours, 24 hours; Sequence 2 predose (0); Sequence 3 predose, 0.5-1 hour; Sequence 4 predose (0); Sequence 5 predose (0); Sequence 6 predose (0) and 2-4 hours; Sequence 7 predose (0), 0.5-1 hr and 4-6 hours. The PK sampling scheme is to continue for subjects who reach the therapeutic dose at early dose levels.
²⁰ In case of QTc prolongation >500 msec or >60 msec from baseline, an additional PK sample will be drawn as close to the observed time of event

### Appendix B Laboratory Parameters

Laboratory parameters to be tested will be at times indicated in the Time and Events Table (Appendix A).

Hematology—Safety

| Platelet Count | RBC Indices: | Automated WBC Differential: |
|---|---|---|
| RBC Count | MCV | Neutrophils |
| WBC Count (absolute) | MCH | Lymphocytes |
| | MCHC | Monocytes |
| Hemoglobin | | Eosinophils |
| Hematocrit | | Basophils |

Clinical Chemistry—Safe

| Blood Urea Nitrogen | Liver Function Tests (LFTs) |
|---|---|
| Creatinine | AST (SGOT) |
| Glucose, fasting | ALT (SGPT) |
| Sodium | GGT |
| Potassium | Alkaline phosphatase |
| Chloride | Total and direct bilirubin |
| Total CO₂ | |
| Calcium, Magnesium, | |
| Phosphate | |
| Uric Acid | |
| Albumin | |
| Total Protein | |

Urinalysis—Safety

Specific gravity
pH, glucose, protein, blood and ketones by dipstick
Microscopic examination (if blood or protein is > trace positive
by dipstick)

Other Parameters (at times specified in the Time and Events Table-Appendix A)

HIV
HbA1c
TSH/free T4
Serum cortisol
ACTH
INR/PT/PTT
Hepatitis B and C
Serum lipid measurements to include: total cholesterol, LDL, HDL,
LDL:HDL ratios, Triglycerides
CRP
Serum testosterone (both men and women)
Pregnancy test (urine)
Spot urine for albumin/creatinine ratio - if normal at baseline, no further
testing required throughout the study
Urinary free cortisol and total creatinine and total volume on 24 hour
urine collections
IGF-1

### Appendix C Features of Cushing's Syndrome

Signs and symptoms of CS include the features listed in the table below; however, they should not be limited to the list. Additional signs and symptoms may be determined by the investigator, as appropriate.

Clinical Features of Cushing's Syndrome^{a}

| | | |
|---|---|---|
| Symptoms | Signs | Overlapping Conditions |

Features that best discriminate CS; most do not have a high sensitivity

Easy bruising
Facial plethora
Proximal myopathy (or proximal
muscle weakness)
Striae (especially if reddish purple
and >1 cm wide)

CS features in the general population that are common and/or less discriminatory

| | | |
|---|---|---|
| Depression | Dorsocervical fat pad (“buffalo | Hypertension^{b} |
| | hump”) | |
| Fatigue | Facial fullness | Incidental adrenal mass |
| Weight gain | Obesity | Vertebral osteoporosis^{b} |
| Back pain | Supraclavicular fullness | Polycystic ovary syndrome |
| Changes in appetite | Thin skin^{b} | Type 2 diabetes |
| Decreased concentration | Peripheral edema | Hypokalemia |
| Decreased libido | Acne | Kidney stones |
| Impaired memory | Hirsutism or female balding | Unusual infections |
| (especially short term) | | |
| Insomnia | Poor skin healing | |
| Irritability | | |
| Menstrual abnormalities | | |

^{a}Features are listed in random order.
^{b}CS is more likely if onset of the feature is at a younger age. Reference: Nieman, et al. The diagnosis of Cushing's syndrome: An Endocrine Society Clinical Practice Guideline. J Clin Endocrin Metab 2008; 98(5): 1526-1540, herein incorporated by reference.

VAS Questionnaire Criteria for Evaluating Physical
Signs of Cushing's Syndrome

| Subject + Physician | Physician Only | Subject Only |
|---|---|---|
| Facial plethora | Proximal myopathy (or | Easy bruising |
| | proximal muscle | |
| | weakness) | |
| Striae (especially if | | Hirsutism or female |
| reddish purple and >1 cm | | balding |
| wide) | | |
| Thin skin | | Poor skin healing |
| Dorsocervical fat pad | | Peripheral edema |
| (“buffalo hump”) | | |
| Facial fullness | | |
| Supraclavicular fullness | | |

The following physical signs of CS will be quantified by the Investigator using specific grading systems included in the SPM:
Acne (Doshi, et al. A comparison of current acne grading systems and proposal of a novel system. Int J Derm. 1997; 36: 416-418, herein incorporated by reference)
Hirsutism (Hatch, et al. Hirsutism: implications, etiology and management. Am J Obstet Gynecol. 1981; 140: 815-830, herein incorporated by reference)
Peripheral edema (Brodovicz, et al. Reliability and Feasibility of Methods to Quantitatively Assess Peripheral Edema. Clin Med Res 2009; 7(½): 21-31, herein incorporated by reference)

### Appendix D Conditions Associated with Pseudo-Cushing's Syndrome

Conditions Associated with Hypercortisolism in the Absence of Cushing's Syndrome

Reference: Nieman, et al. The diagnosis of Cushing's syndrome: An Endocrine Society Clinical Practice Guideline. J Clin Endocrin Metab 2008; 98(5): 1526-1540, herein incorporated by reference. 48 hour, 2 mg dexamethasone suppression test may be necessary to exclude pseudo-Cushing's disease. Normal cortisol ≤1.4 μg/dL.

Some clinical features of CS may be present:
Pregnancy
Depression and other psychiatric conditions
Alcohol dependence
Glucocorticoid resistance
Morbid obesity
Poorly controlled diabetes mellitus

Unlikely to have any clinical features of CS
Physical stress (hospitalization, surgery, pain)
Malnutrition, anorexia nervosa
Intense chronic exercise
Hypothalamic amenorrhea
Cortisol-binding globulin (CBG) excess (increased serum but not urine cortisol)

### Appendix E Testing Criteria for Diagnosis of Diabetes Mellitus

The American Diabetes Association recommendations state that pre-diabetes (impaired glucose tolerance) and diabetes can be provisionally diagnosed with any one of the following criteria:

### Pre-Diabetes/Impaired Glucose Tolerance:

3. A fasting glucose of ≥100 and <126 mg/dL (after no caloric intake for at least 8 hours)

Diabetes
7. A fasting glucose of ≥126 mg/dL (after no caloric intake for at least 8 hours) OR
8. A casual measurement of glucose ≥200 mg/dL (taken any time of day without regard to time of last meal) with classic diabetes symptoms: increased urination, increased thirst and unexplained weight loss OR
9. A glucose level on an oral glucose tolerance test (OGTT) (75 gram dose) of ≥200 mg/dL for the 2 hour sample. Oral glucose tolerance testing is not necessary if the subject has a glucose level of ≥126 mg/dL.

In the absence of unequivocal hyperglycemia with acute metabolic decompensation, the diagnosis should be confirmed on a subsequent day, by any of the above 3 criteria.

### Appendix F Guidelines for Hypertension

Reference: The Seventh Report of the Joint National Committee on Prevention, Detection, Evaluation, and Treatment of High Blood Pressure (JNC 7), 2004, herein incorporated by reference.

Classification of Blood Pressure Levels for Adults >18 Years of Age

| Blood Pressure Classification | SBP (mmHg) | DBP (mmHg) |
|---|---|---|
| Normal | <120 | and <80 |
| Pre-hypertension | 120-139 | or 80-89 |
| Stage 1 hypertension | 140-159 | or 90-99 |
| Stage 2 hypertension | ≥160 | or ≥100 |

Recommendations for Follow-Up Based on Initial Blood Pressure Measurements for Adults without Acute End Organ Damage

| Initial | |
|---|---|
| Blood Pressure (mmHg)^{a} | Follow-up Recommended^{b} |
| Normal | Recheck in 2 years |
| Pre-hypertension | Recheck in 1 year^{c} |
| Stage 1 hypertension | Confirm within 2 months^{c} |
| Stage 2 hypertension | Evaluate or refer to source of care within 1 |
| | month. For those with higher pressures |
| | (eg. >180/110 mmHg), evaluate and treat |
| | immediately or within 1 week depending on |
| | clinical situation and complications. |

^{a}If systolic and diastolic categories are different, follow recommendations for shorter time follow-up (eg. 160/86 mmHg should be evaluated or referred to source of care within 1 month)
^{b}Modify the scheduling of follow-up according to reliable information about past BP measurements, other cardiovascular risk factors, or target organ disease
^{c}Provide advice about lifestyle modifications.

### Appendix G CyP450 3A4 Drug Interactions

Contact Sponsor if a Subject Will be on any of these Drugs

| | | Agents that can | Agents that can |
|---|---|---|---|
| | | increase exposure to | decrease exposure to |
| | | the 2S,4R | the 2S,4R |
| Agents that can be affected by the 2S,4R | | ketoconazole | ketoconazole |
| ketoconazole enantiomer | | enantiomer | enantiomer |
| Macrolide | Miscellaneous: | indinavir | efavirenz |
| antibiotics: | alfentanil | nelfinavir | nevirapine |
| clarithromycin | aprepitant | ritonavir | barbiturates |
| erythromycin | aripiprazole | clarithromycin | carbamazepine |
| NOT azithromycin | boceprevir | itraconazole | glucocorticoids |
| telithromycin | buspirone | ketoconazole | modafinil |
| Anti-arrhythmics: | cafergot | nefazodone | oxcarbazepine |
| quinidine->3-0H | caffeine | saquinavir | phenobarbital |
| Benzodiazepines: | cilostazol | telithromycin | phenytoin |
| alprazolam | cocaine | aprepitant | pioglitazone |
| diazepam--30H | codeine- | erythromycin | rifabutin |
| midazolam | N-demethylation | fluconazole | rifampin |
| triazolam | dapsone | grapefruit or juice | St. John's wort |
| Immune | dexamethasone | blood orange or juice | troglitazone |
| Modulators: | dextromethorphan | verapamil | |
| cyclosporine | docetaxel | diltiazem | |
| tacrolimus | domperidone | cimetidine | |
| HIV Antivirals: | eplerenone | amiodarone | |
| indinavir | fentanyl | NOT azithromycin | |
| nelfinavir | finasteride | chloramphenicol | |
| ritonavir | gleevec | boceprevir | |
| saquinavir | haloperidol | ciprofloxacin | |
| Prokinetic: | irinotecan | delaviridine | |
| cisapride | LAAM | diethyl- | |
| Antihistamines: | lidocaine | dithiocarbamate | |
| asternizole | methadone | fluvoxamine | |
| chlorpheniramine | nateglinide | gestodene | |
| terfenadine | ondansetron | imatinib | |
| Calcium Channel | pimozide | mibefradil | |
| Blockers: | propranolol | mifepristone | |
| amlodipine | quetiapine | norfloxacin | |
| diltiazem | quinine | norfluoxetine | |
| felodipine | risperidone | starfruit | |
| lercanidipine | salmeterol | telaprevir | |
| nifedipine nisoldipine | sildenafil | voriconazole | |
| nitrendipine | sirolimus | | |
| verapamil | sorafenib | | |
| HMG CoA | sunitinib | | |
| Reductase | tamoxifen | | |
| Inhibitors: | taxol | | |
| atorvastatin | telaprevir | | |
| cerivastatin | terfenadine | | |
| lovastatin | torisel | | |
| NOT pravastatin | trazodone | | |
| NOT rosuvastatin | vincristine | | |
| simvastatin | zaleplon | | |
| Steroid 6beta-OH: | ziprasidone | | |
| estradiol | zolpidem | | |
| hydrocortisone | | | |
| progesterone | | | |
| testosterone | | | |

### Appendix H Special Considerations

| Disease | Symptoms | Signs | Laboratory values |
|---|---|---|---|
| Hypocortisolemia | Fatigue | Low blood pressure | Reduced serum and |
| | Muscle weakness | Symptomatic | salivary cortisol |
| | Loss of appetite | Orthostatic | levels |
| | Weight loss | hypotension | Hypoglycemia |
| | Nausea, vomiting | Reduction in weight | |
| | Dizziness, esp. on | | |
| | standing | | |
| | Irritability | | |
| | Depression | | |
| | Sweating | | |
| | Joint aches and | | |
| | pains | | |
| Hypomineralocorticoidism | Muscle weakness | Low blood pressure | Hyperkalemia |
| | Fatigue | Severe orthostatic | Hyponatremia |
| | Fainting | hypotension | |
| | Salt craving | | |
| | Irritability | | |
| Hypogonadism | Erectile dysfunction | Gynecomastia | reduced testosterone |
| | Reduction in beard | Reduced body hair | levels (AE in males, |
| | and body hair | Osteoporosis | beneficial in |
| | Enlarged breasts (in | | women) |
| | men) | | |
| | Fatigue | | |
| | Reduced libido | | |
| | Hot flashes | | |
| | Difficulty | | |
| | concentrating | | |

What is claimed is:

1. A method for treatment of Cushing's Syndrome in a patient in need thereof comprising administering a 2S,4R ketoconazole enantiomer substantially free of the 2R,4S enantiomer to the patient according to a dosing titration schedule comprising a first dosing level, said first dosing level comprising administering 150 to 300 milligrams of the 2S,4R ketoconazole enantiomer to the patient per day in 1 to 3 divided doses.

2. The method of claim 1 wherein the first dosing level comprises administering 150 milligrams of the 2S,4R ketoconazole enantiomer to the patient two times a day.

3. The method of claim 1 wherein the first dosing level is administered for about 8 to about 22 days.

4. The method of claim 1 wherein the first dosing level is administered for about 15 days.

5. The method of claim 1 further comprising measuring the patient's mean 24-hour urinary free cortisol levels, wherein a subsequent dosing level is administered if the mean 24-hour urinary free cortisol levels are greater than an upper limit of the normal range established for an assay being used at a central laboratory, and wherein the first dosing level or the subsequent dosing level is maintained if one or more of the following apply: (1) mean 24-hour urinary free cortisol levels are less than the upper limit of the normal range established for the assay being used at a central laboratory; (2) the maximum allowed dose is reached; or (3) the highest tolerated dose is reached.

6. The method of claim 5 further comprising a second dosing level, said second dosing level comprising administering 450 milligrams of the 2S,4R ketoconazole enantiomer to the subject per day in 1 to 3 divided doses.

7. The method of claim 6 further comprising a third dosing level, said third dosing level comprising administering 600 milligrams of the 2S,4R ketoconazole enantiomer to the subject per day in 1 to 3 divided doses.

8. The method of claim 7 further comprising a fourth dosing level, said fourth dosing level comprising administering 750 milligrams of the 2S,4R ketoconazole enantiomer to the subject per day in 1 to 3 divided doses.

9. The method of claim 8 further comprising a fifth dosing level, said fifth dosing level comprising administering 900 milligrams of the 2S,4R ketoconazole enantiomer to the subject per day in 1 to 3 divided doses.

10. The method of claim 9 further comprising a sixth dosing level, said sixth dosing level comprising administering 1050 milligrams of the 2S,4R ketoconazole enantiomer to the subject per day in 1 to 3 divided doses.

11. The method of claim 10 further comprising a seventh dosing level, said seventh dosing level comprising administering 1200 milligrams of the 2S,4R ketoconazole enantiomer to the subject per day in 1 to 3 divided doses.
